(19) United States
(12) Patent Application Publication
(10) Pub. No.: US 2023/0178243 A1
(43) Pub. Date: Jun. 8, 2023

(54) PREDICTING A RECOMMENDED THERAPY FROM GUT COMPOSITIONAL DATA

(71) Applicant: Institute for Systems Biology, Seattle, WA (US)

(72) Inventors: Tomasz Wilmanski, Seattle, WA (US); Noa Rappaport, Bellevue, WA (US); Andrew T. Magis, Seattle, WA (US); Sean M. Gibbons, Seattle, WA (US)

(73) Assignee: Institute for Systems Biology, Seattle, WA (US)

(21) Appl. No.: 18/160,678

(22) Filed: Jan. 27, 2023

Related U.S. Application Data
(63) Continuation-in-part of application No. 18/060,382, filed on Nov. 30, 2022, now abandoned.
(60) Provisional application No. 63/328,862, filed on Apr. 8, 2022.
(60) Provisional application No. 63/264,753, filed on Dec. 1, 2021.

(51) Int. Cl. G16H 50/20; G16B 40/20; C12Q 1/689; G16H 10/40; G16H 40/67

(52) U.S. Cl. CPC G16H 50/20; C12Q 1/689; G16B 40/20; G16H 10/40; G16H 40/67

(57) ABSTRACT

Predicting therapy from gut compositional data is described herein. In an example, a system accesses gut compositional data including a taxonomic abundance, a taxonomic diversity, and/or an enterotype for a subject. The system generates a gut microbiome signature for a safety and an efficacy of a statin therapy for the subject by applying a classifier to the gut compositional data. The safety of the statin therapy is characterized by an insulin resistance of the subject and the efficacy of the statin therapy is characterized by a blood hydroxymethylglutarate level of the subject. The system determines a recommended therapy for the subject based on the gut microbiome signature and one or more taxa of the gut compositional data of the subject. The recommended therapy is selected from a statin therapy intensity, a probiotic therapy, a prebiotic therapy, or a combination thereof. The system outputs the recommended therapy.

### CROSS-REFERENCE TO RELATED APPLICATIONS

This application is a continuation-in-part of U.S. Serial No. 18/060,382, filed Nov. 30, 2022, which claims the benefit of and priority to U.S. Provisional Application No. 63/264,753, filed on Dec. 1, 2021. This application also claims the benefit of and priority to U.S. Provisional Application No. 63/328,862, filed Apr. 8, 2022. Each of these applications is hereby incorporated by reference in its entirety for all purposes.

### STATEMENT REGARDING FEDERALLY SPONSORED RESEARCH OR DEVELOPMENT

This invention was supported by the M.J. Murdock Charitable Trust, WRF Distinguished Investigator Award, National Academy of Medicine Catalyst Award and the NIH grant (no. U19AG023122) awarded by the NIA). The government has certain rights in the invention.

### FIELD

Embodiments relate to generating a recommended therapy by using a classifier to process gut compositional data. The gut compositional data may include one or more attributes that correspond to a given subject.

### BACKGROUND

Statins are a group of medications commonly prescribed for the purpose of treating or preventing atherosclerotic cardiovascular disease (ACVD). While statins are effective in decreasing ACVD-associated mortality, considerable heterogeneity exists in terms of efficacy of lowering low-density lipoprotein (LDL) cholesterol. Furthermore, statin use can give rise to a number of adverse side effects in a subset of subjects. These side effects can include myopathy, disrupted glucose control, and an increased risk of developing type II diabetes (T2D). Several guidelines exist for which at-risk populations are prescribed statins and at what intensity. However, despite considerable progress in identifying pharmacological and genetic factors contributing to heterogeneity in statin response, personalized approaches to statin therapy remain limited.

Therefore, it would be advantageous to monitor and process pertinent indicators to predict a recommended therapy, particularly related to a statin therapy intensity, so as to facilitate treatment that may result in better outcomes for a subject.

### SUMMARY

Embodiments of the present disclosure relate to using a classifier to process gut compositional data to generate a recommended therapy for a subject. In some embodiments, a computer-implemented method is provided that involves (a) accessing gut compositional data including a taxonomic abundance, a taxonomic diversity, and/or an enterotype for a subject; (b) generating a gut microbiome signature for a safety of a statin therapy for the subject and an efficacy of the statin therapy for the subject by applying a classifier to the gut compositional data, the safety of the statin therapy characterized by an insulin resistance of the subject, and the efficacy of the statin therapy characterized by a blood hydroxymethylglutarate (HMG) level of the subject; (c) determining a recommended therapy for the subject based on the gut microbiome signature and one or more taxa of the gut compositional data of the subject, the recommended therapy selected from a statin therapy intensity, a probiotic therapy, a prebiotic therapy, or a combination thereof; and (d) outputting the recommended therapy.

In some embodiments, determining the recommended therapy involves comparing the gut microbiome signature and the gut compositional data of the subject to a reference dataset. The reference dataset includes a plurality of gut microbiome data and blood metabolite data of a reference population exhibiting variable insulin resistance and blood HMG level responses to a given statin therapy intensity.

In some embodiments, the computer-implemented method further involves determining a presence of Akkermansia for the subject is below a first threshold based on the gut compositional data and facilitating the probiotic therapy and/or the prebiotic therapy for the subject based on the presence of Akkermansia being below the first threshold.

In some embodiments, the computer-implemented method further involves determining the blood HMG level for the subject; and generating the gut microbiome signature for the subject by applying the classifier to the gut compositional data and the blood HMG level.

In some embodiments, the computer-implemented method further involves accessing fecal nucleic acid sequence data and/or blood metabolite data for the subject; and generating the gut compositional data for the subject based on the fecal nucleic acid sequence data and/or the blood metabolite data.

In some embodiments, the computer-implemented method further involves determining the recommended therapy by performing one or more steps selected from determining the gut compositional data includes a relative abundance of Bacteroides ssp. above a first threshold for the subject; determining that the enterotype included in the gut compositional data is a Bacteroides 1 enterotype or a Bacteroides 2 enterotype; determining the gut compositional data includes an alpha-diversity below a second threshold for the subject; and determining the statin therapy intensity is below a threshold intensity.

In some embodiments, the computer-implemented method further involves determining the recommended therapy by performing one or more steps selected from determining the gut compositional data includes a relative abundance of Bacteroides ssp. above a first threshold for the subject; determining that the enterotype included in the gut compositional data is a Bacteroides 1 enterotype or a Bacteroides 2 enterotype; determining the gut compositional data includes an alpha-diversity below a second threshold for the subject; determining at least one of: (i) a presence of Akkermansia for the subject, (ii) an insulin resistance characterization for the subject, or (iii) a treatment for insulin resistance for the subject; and determining the statin therapy intensity is above a threshold intensity.

In some embodiments, the computer-implemented method further involves determining the recommended therapy by performing one or more steps selected from determining the gut compositional data includes a relative abundance of Bacteroides ssp. below a first threshold for the subject; determining that the enterotype indicated by the gut compositional data excludes a Bacteroides enterotype; determining the gut compositional data includes an alpha-diversity greater than a second threshold for the subject; and determining a statin therapy intensity is greater than a threshold intensity.

In some embodiments, the computer-implemented method further involves determining a genetic risk score associated with the subject having one or more alleles associated with the efficacy of the statin therapy for the subject or the safety of the statin therapy for the subject; and generating the gut microbiome signature for the subject by applying the classifier to the gut compositional data and the genetic risk score.

In some embodiments, a system is provided that includes one or more data processors and a non-transitory computer readable storage medium containing instructions which, when executed on the one or more data processors, cause the one or more data processors to perform a set of actions including (a) accessing gut compositional data including a taxonomic abundance, a taxonomic diversity, and/or an enterotype for a subject; (b) generating a gut microbiome signature for a safety of a statin therapy for the subject and an efficacy of the statin therapy for the subject by applying a classifier to the gut compositional data, the safety of the statin therapy characterized by an insulin resistance of the subject, and the efficacy of the statin therapy characterized by a blood hydroxymethylglutarate (HMG) level of the subject; (c) determining a recommended therapy for the subject based on the gut microbiome signature and one or more taxa of the gut compositional data of the subject, the recommended therapy selected from a statin therapy intensity, a probiotic therapy, a prebiotic therapy, or a combination thereof; and (d) outputting the recommended therapy.

In some embodiments, a computer-program product is provided that is tangibly embodied in a non-transitory machine-readable storage medium and that includes instructions configured to cause one or more data processors to perform a set of actions including (a) accessing gut compositional data including a taxonomic abundance, a taxonomic diversity, and/or an enterotype for a subject; (b) generating a gut microbiome signature for a safety of a statin therapy for the subject and an efficacy of the statin therapy for the subject by applying a classifier to the gut compositional data, the safety of the statin therapy characterized by an insulin resistance of the subject, and the efficacy of the statin therapy characterized by a blood hydroxymethylglutarate (HMG) level of the subject; (c) determining a recommended therapy for the subject based on the gut microbiome signature and one or more taxa of the gut compositional data of the subject, the recommended therapy selected from a statin therapy intensity, a probiotic therapy, a prebiotic therapy, or a combination thereof; and (d) outputting the recommended therapy.

### BRIEF DESCRIPTION OF THE DRAWINGS

The present disclosure is described in conjunction with the appended figures:

FIG. 1 shows an exemplary computing system for predicting a recommended therapy from gut compositional data according to some aspects of the present disclosure;

FIG. 2 illustrates an exemplary process of predicting a recommended therapy from gut compositional data according to some aspects of the present disclosure;

FIG. 3 illustrates exemplary results of plasma hydroxymethylglutarate as a marker of statin use and efficacy;

FIG. 4 illustrates exemplary results of gut microbiome composition modifying statin efficacy;

FIG. 5 illustrates exemplary results of gut alpha-diversity being anti-correlated with markers of statin on-target effects;

FIG. 6 illustrates exemplary results of microbiome enterotypes modifying statin efficacy and metabolic side effects;

FIG. 7 illustrates exemplary results of enterotypes differing in their relative abundance of short-chain fatty acid-producing taxa;

FIG. 8 illustrates exemplary results of microbiome enterotypes modifying markers of statin on- and off-target effects;

FIG. 9 illustrates exemplary results of Shannon diversity biomarkers predicting hydroxymethylglutarate levels exclusively in statin users;

FIG. 10 illustrates exemplary results of blood metabolomics data predicting a Bacteroides 2 enterotype; and

FIG. 11 illustrates exemplary results of Bacteroides abundance predicting insulin resistance feature levels exclusively in statin users and including a presence of Akkermansia in an insulin resistance risk score.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### Overview

Typically, treatment decisions for statin therapies are made through trial-and-error between a clinician and a subject to obtain an optimal tolerable dose. Avoiding this trial-and-error phase through individualized analysis of genetic, physiological, and health parameters can improve medication tolerance, adherence, and long-term health benefits, as well as guide complementary therapies aimed at mitigating side effects.

Similar to other prescription medications, statins are widely metabolized by gut bacteria into secondary compounds. This indicates that the gut microbiome may impact statin bioavailability or potency to its host, contributing to the interindividual variability in low-density lipoprotein (LDL) response seen among statin users. Additionally, biochemical modification of statins by gut bacteria could potentially contribute to side effects of the drug. Independent of statins, the gut microbiome contributes to host metabolic health through regulating insulin sensitivity, blood glucose, and inflammation, hence sharing considerable overlap with off-target effects of statin therapy.

Some embodiments relate to using gut compositional data of a subject to determine a recommended therapy. The gut compositional data represents microbiome information about the gut of the subject and may include one or more of a taxonomic abundance of the subject, a taxonomic diversity of the subject, or an enterotype of the subject. The recommended therapy may be a statin therapy intensity, a probiotic therapy, a prebiotic therapy, or a combination thereof.

The gut compositional data may be derived through 16S ribosomal ribonucleic acid (RNA) amplicon or shotgun metagenomic sequencing of a stool sample, blood markers for gut microbiome composition, or both, regardless of whether a subject is taking a statin. The gut compositional data therefore generally includes or is selected from fecal nucleic acid sequence data, blood metabolite data, or a combination of the fecal nucleic acid sequence data and the blood metabolite data.

One embodiment provides a method for predicting a recommended therapy for a subject that involves accessing gut compositional data including a taxonomic abundance, a taxonomic diversity, and/or an enterotype for a subject. A classifier is applied to the gut compositional data to generate a gut microbiome signature for a safety (e.g., a risk of the subject experiencing side effects related to insulin resistance) of a statin therapy for the subject and an efficacy of the statin therapy for the subject. The efficacy of the statin therapy is characterized by a blood hydroxymethylglutarate (HMG) level of the subject. A recommended therapy for the subject is determined based on the gut compositional data (e.g., taxonomic abundance, a taxonomic diversity, and/or an enterotype) and one or more taxa (e.g., Bacteroides, Prevotella, Ruminococcus, Akkermansia, and/or SCFA-producing commensals such as Faecalibacterium and Subdoligranulum) of the subject. The recommended therapy may be a statin therapy intensity, a probiotic therapy, a prebiotic therapy, or a combination thereof. The recommended therapy is output and the recommended therapy can be facilitated for the subject.

Facilitating the recommended therapy may involve generating a recommendation for providing the statin therapy intensity to the subject. The recommendation can indicate a dosage for the statin therapy or a range of dosages for the statin based on the recommended therapy. The recommendation may additionally include supporting information that is indicative as to why the recommendation is provided. In some instances, particular gut compositional data (e.g., a high alpha-diversity) may be associated with a lower efficacy and a higher insulin resistance. As a result, the recommended therapy may involve recommending a higher dosage since the subject may be less likely to experience side effects. Conversely, other gut compositional data may be associated with a higher efficacy and a lower insulin resistance, so the recommended therapy may involve a recommendation of a lower dosage since side effects (e.g., a development of diabetes) may be more likely to occur for the subject. Low Akkermansia, which can be determined from the gut compositional data, along with a lower statin efficacy (e.g., as indicated by HMG levels) or a higher insulin resistance, may result in an additional therapy being recommended for the subject to increase the statin efficacy or to increase the safety of the statin therapy. For instance, a probiotic therapy or a prebiotic therapy designed to increase Akkermansia may be determined as the recommended therapy.

The statin therapies, include, but are not limited to, Pitavastatin, Lovastatin, Pravastatin, Simvastatin, Atorvastatin, and Rosuvastatin. In an example, the target statin therapy may be characterized as a low intensity, a moderate intensity, or a high intensity. A low intensity may involve a daily treatment with 1 milligram (mg) of Pitavastatin, 20 mg of Lovastatin, 10 to 20 mg of Pravastatin, or 10 mg of Simvastatin. In another example, the low intensity statin therapy may involve daily treatment with 2.5 to 5 mg of Atorvastatin or 1.5 to 2.5 mg of Rosuvastatin. A moderate intensity may involve daily treatment with 2 to 4 mg of Pitavastatin, 40 to 80 mg of Lovastatin, 40 to 80 mg of Pravastatin, 20 to 40 mg of Simvastatin, 10 to 20 mg of Atorvastatin, or 5 to 10 mg of Rosuvastatin 5 to 10 mg. A high intensity may involve daily treatment with 40 to 80 mg of Atorvastatin or 20 to 40 mg of Rosuvastatin.

Statin efficacy and safety, as measured by blood HMG levels and assessment of insulin resistance, respectively, is directly impacted by the gut microbiome. As an example, a subject having a Bacteroides enterotype, low alpha-diversity, genetic markers that modify statin response, and/or a high Bacteroides abundance without Akkermansia may exhibit the greatest increases in blood HMG levels and insulin resistance with statin use. Since HMG levels also reflect on-target and off-target effects not captured by other markers such as LDL-cholesterol, HMG levels afford time-invariant accounting of on-target statin efficacy, whereas LDL-cholesterol requires knowledge of pre-statin cholesterol levels to calculate the percent decrease in LDL over time. HMG levels also provide insight into statin off-target effects obscured by statin on-target variability. So, determining a recommended therapy for a subject based on gut compositional data and statin efficacy may provide improved treatment compared to the typical approaches of using LDL levels and trial-and-error.

### Definitions

“Enterotype” refers to classification of an individual based on the bacteriological composition of their gut microbiota. A Bacteroides (“Bac.”) enterotype is characterized by high frequency or relative abundance of Bacteroides genus. A Prevotella (“Prev.”) enterotype is characterized by low frequency of Bacteroides genus but high relative frequency of Prevotella genus. A Ruminococcus (“Rum.”) enterotype has a high frequency of Ruminococcus genus enriched for taxa primarily from the Firmicutes phylum as well as Akkermansia. Classification of the Bacteroides enterotype can be further subdivided further into Bacteroides 1 (“Bac.1”) and Bacteroides 2 (“Bac.2”), with the Bac. I enterotype being characterized by high Bacteroides genus frequency and high Faecalibacterium prausnitzii frequency, and with the Bac.2 enterotype being characterized by high Bacteroides genus and low Faecalibacterium prausnitzii frequency. Enterotyping can be carried out with taxon-based and cluster-based classifiers. An example enterotyping method is Dirichlet Multinomial Mixture (DMM) modeling on the rarefied genus-level count data.

“Taxonomic abundance” refers to relative abundance profiles of individual taxonomic strata (e.g., domain, kingdom, phylum, class, order, family, genus, species, and sub-species strata: (e.g.,. operational taxonomic units, amplicon sequence variants, strains, etc.)), estimated from amplicon or shotgun-metagenomic sequencing data. An example is Bacteroides ssp. abundance, which refers to either the combined or individual relative abundances of species within the genus Bacteroides in a given sample.

“Taxonomic diversity” refers to the number of taxonomic features in a sample and to the evenness of the abundance distribution (e.g., a greater number of features and greater evenness contribute to higher taxonomic diversity). An example is the Shannon Diversity Index or Shannon diversity, which refers to the Shannon entropy of a relative abundance distribution and takes both number of taxonomic features and the evenness of the abundance distribution into account.

### Systems and Methods for Statin Therapy Intensity Prediction

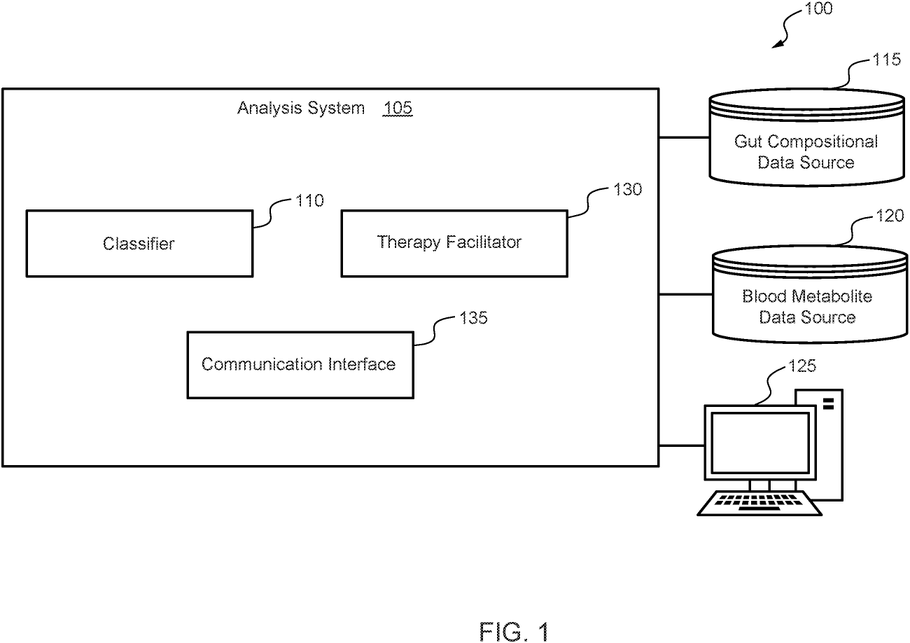

FIG. 1 shows an exemplary computing system 100 for facilitating identification of a recommended therapy based on gut compositional data. The computing system 100 can include an analysis system 105 to execute a classifier 110 for determining a gut microbiome signature. The classifier 110 may be rule-based or may include a machine-learning model. Examples of the machine-learning model include a decision tree, k-nearest neighbor model, a logistic regression model, etc. The machine-learning model may be trained and/or used to (for example) predict a gut microbiome signature from which a recommended therapy for a subject can be determined.

In some instances, if the classifier 110 is a machine-learning model, the classifier 110 may be trained using training data of one or more training data sets. Each training data set of the can include a set of training data for subjects on and off statins. The training data can include blood HMG levels of the subjects. In addition, the training data can include a taxonomic abundance of the subjects, a taxonomic diversity of the subjects, and/or an enterotype of the subjects. In some instances, the training data may further include blood insulin levels of the subjects, blood glucose levels of the subjects, blood hemoglobin Alc (HbAlc) levels of the subjects, blood LDL-cholesterol levels of the subjects, and/or Homeostatic Model Assessment for Insulin Resistance (HOMA-IR) of the subjects. Each subject in a first subset of the set of training data may be associated with a low statin therapy intensity for the subject, each subject in a second subset of the set of training data may be associated with a moderate statin therapy intensity for the subject, and each subject in a third subset of the set of training data may be associated with a high statin therapy intensity for the subject. The training data may have been collected (for example) from one or more data sources, such as a gut compositional data source 115 that stores gut compositional data for subjects and a blood metabolite data source 120 that stores blood metabolite data for subjects.

The computing system 100 can map the training data associated with a low statin efficacy intensity to a “low efficacy” label, the training data associated with a high statin efficacy to a “high efficacy” label, the training data associated with a low safety to a “low safety” label, and the training data associated with a high safety to a “high safety” label. Additional labels may associate training data to statin therapy intensities. Mapping data may be stored in a mapping data store (not shown). The mapping data may identify each subject that is mapped to each of the labels. In some instances, labels associated with the training data may have been received or may be derived from data received from one or more provider systems 125, each of which may be associated with (for example) a user, nurse, treatment facility, etc. associated with a particular subj ect.

The analysis system 105 can use the mappings of the training data to train the classifier 110. More specifically, the analysis system 105 can access an architecture of a model, define (fixed) hyperparameters for the model (which are parameters that influence the learning rate, size, and complexity of the model, etc.), and train the model such that a set of parameters are learned. More specifically, the set of parameters may be learned by identifying parameter values that are associated with a low or lowest loss, cost or error generated by comparing predicted outputs (obtained using given parameter values) with actual outputs.

Once trained, the classifier 110 can use the architecture and learned parameters to process non-training data and generate a result. For example, classifier 110 may access an input data set that includes gut compositional data for a subject. In some instances, the analysis system 105 may generate the gut compositional data by accessing fecal nucleic acid sequence data or blood metabolite data for the subject. The analysis system 105, or another system (e.g., the provider system 125) can perform 16S RNA amplicon or shotgun metagenomic sequencing on a stool sample of the subject to determine the fecal nucleic acid sequence data. Additionally or alternatively, the analysis system 105 may determine blood markers for gut microbiome composition in blood metabolite data for the subject received from the blood metabolite data source 120.

In some instances, the input data set accessed by the classifier 110 can include a blood HMG level of the subject, a genetic risk score for the subject, and/or a statin therapy status for the subject (e.g., whether a subject is currently undergoing statin therapy). The blood HMG level may be obtained from the blood metabolite data or from the provider system 125 based on an assessment performed by a clinician. The genetic risk score can be associated with the subject having one or more alleles associated with the efficacy of the statin therapy for the subject or the safety of the statin therapy for the subject. For instance, certain single nucleotide polymorphisms (SNP) are associated with a higher statin efficacy and/or a higher risk of side effects related to insulin resistance. So, a genetic sequence of the subject can be determined or accessed by the analysis system 105 to determine the genetic risk score. As a particular example, the presence of rs445925 or rs7412 may be associated with a higher statin efficacy.

The input data set can be fed into a machine-learning model having an architecture used during training and configured with learned parameters. The machine-learning model can output a prediction of a gut microbiome signature for the subject. The gut microbiome signature may represent a safety of a statin therapy for the subject and an efficacy of the statin therapy for the subject. The safety is characterized by an insulin resistance of the subject and the efficacy is characterized by a blood HMG level of the subject.

The prediction of the gut microbiome signature along with one or more taxa of the gut compositional data can be used by the analysis system 105 to determine a recommended therapy for the subject. For instance, the recommended therapy may indicate whether a low intensity, moderate intensity, or high intensity for a statin therapy is recommended for the subject based on the gut microbiome signature and the gut compositional data (and optionally additional features). The recommended therapy may include other characterizations and/or levels of therapy intensity. For instance, the recommended therapy may be numerical (e.g., between 0 and 5), with a lower number representing a lower intensity for the statin therapy. The therapy facilitator 130 may additionally facilitate an additional therapy based on the gut compositional data or the output of the classifier 110. The recommended therapy may additionally or alternatively include a recommendation to treat the subject with a composition including a cardio-metabolic probiotic, such as Akkermansia muciniphila, or a prebiotic that encourages growth of Akkermansia. The classifier 110 can output the recommended therapy.

In some instances, the classifier 110 can be rule-based. So, the classifier 110 can include one or more rule sets that each include a first rule characterizing the gut compositional data of the subject and a second rule indicating the recommended therapy according to the gut compositional data. The classifier 110 may compare the gut microbiome signature and the gut compositional data of the subject to a reference dataset that includes gut microbiome and blood metabolite data of a reference population exhibiting variable insulin resistance and blood HMG level responses to statin therapy intensity. As an example, the gut compositional data can indicate a relative abundance of Bacteroides ssp. for the subject, an enterotype for the subject, and an alpha-diversity for the subject. In general, the target therapy intensity may be inversely proportional to relative Bacteroides spp. abundance, dependent on Bacteroides enterotype assignment (e.g., whether the gut microbiome is assigned a Bacteroides enterotype or a different one such as a Ruminococacceae or Prevotella enterotype), and directly proportional to the taxonomic diversity.

In some instances, the classifier 110 can determine that gut compositional data including a higher Bacteroides abundance, lower taxonomic diversity, and a Bacteroides enterotype assignment is associated with a lower statin therapy intensity due to a higher statin efficacy and a lower statin safety predicted for the subject and indicated by the gut microbiome signature. Statin efficacy can be characterized by HMG levels of the subject and safety can be characterized by insulin resistance of the subject. A lower insulin resistance may be associated with a higher risk of side effects (e.g., developing diabetes) for the subject when taking a statin therapy. Conversely, the classifier 110 can determine that gut compositional data including a lower Bacteroides abundance, higher taxonomic diversity, and an enterotype assignment other than Bacteroides such as a Ruminococacceae or Prevotella enterotype, is associated with a higher statin therapy intensity due to a lower statin efficacy and a higher statin safety predicted for the subject. As a particular example, classifier 110 may include rule sets that identify the recommended therapy of a statin therapy intensity as being greater than a threshold intensity (e.g., a moderate intensity to a high intensity) if the gut compositional data indicates that the relative abundance of Bacteroides ssp. is below a threshold (e.g., 11.5%), the enterotype is not a Bacteroides enterotype, and/or that the alpha-diversity is greater than a threshold (e.g., 4.47 Shannon Index). As another example, the classifier 110 may determine that the recommended therapy of a statin therapy intensity is below a threshold intensity (e.g., a low intensity to a moderate intensity) if the gut compositional data indicates that the relative abundance of Bacteroides ssp. is above a threshold (e.g., 11.5%), the enterotype is a Bacteroides 1 enterotype or a Bacteroides 2 enterotype, and/or that the alpha-diversity is below a threshold (e.g., 4.47 Shannon Index).

The classifier 110 may adjust the gut microbiome signature based on the gut compositional data indicating a presence or absence of certain attributes. For instance, the classifier 110 may adjust the gut microbiome signature based on the presence or absence of cardio-metabolically relevant gut commensals such as Akkermansia spp., genetic markers for insulin resistance, ongoing monitoring for insulin resistance, and ongoing treating for insulin resistance. An example of monitoring for insulin resistance include, but are not limited to, measuring blood glucose levels. Examples of treating for insulin resistance include, but are not limited to, metformin therapy, glucagon-like protein-1 (GLP-1) receptor agonist therapy, insulin therapy, and cardio-metabolic probiotic therapy. In an example, the classifier 110 can adjust the recommended therapy for a subject with gut compositional data indicating a higher Bacteroides abundance, lower taxonomic diversity, and a Bacteroides enterotype assignment to increase the recommended statin therapy intensity to a maximum intensity by monitoring for insulin resistance and/or treating for insulin resistance. Similarly, the recommended therapy can be adjusted when a higher Bacteroides abundance is indicated by the gut compositional data in combination with the presence of a cardio-metabolically healthy commensal such as Akkermansia. As a particular example, the classifier 110 may determine that the recommended statin therapy intensity is above a threshold intensity (e.g., a moderate intensity to a high intensity) if the gut compositional data indicates that the relative abundance of Bacteroides ssp. is above a threshold (e.g., 11.5%), the enterotype is a Bacteroides 1 enterotype or a Bacteroides 2 enterotype, that the alpha-diversity is below a threshold (e.g., 4.47 Shannon Index), and/or at least one of: (i) a presence of Akkermansia for the subject, (ii) an insulin resistance characterization (e.g., based on measured blood glucose levels) for the subject, or (iii) a treatment for insulin resistance for the subject (e.g., the subject undergoing insulin therapy).

The classifier 110 may additionally account for the blood HMG level, a genetic risk score, and/or a statin therapy status of the subject when determining the recommended therapy. For instance, the classifier 110 may determine the recommended therapy to be a statin therapy of a low intensity to moderate intensity, or high intensity in combination with monitoring and/or treating for insulin resistance, when the subject is characterized as having elevated HMG levels and the gut compositional data indicates the elevated HMG levels. The HMG level can be measured for the subject relative to HMG levels measured in the reference population. Elevated HMG levels are indicative of higher statin efficacy and higher risk of side effects related to insulin resistance. For a genetic risk score indicating that the subject includes one or more alleles associated a higher statin efficacy (e.g., rs445925 or rs7412), the classifier 110 may determine the statin therapy intensity to be between a low intensity to a moderate intensity. In contrast, for a genetic risk score indicating that the subject does not include one or more alleles associated with a higher statin efficacy, the classifier 110 may determine the statin therapy intensity to be between a moderate intensity and a high intensity.

A therapy facilitator 130 of the analysis system 105 can then facilitate a therapy for the subject in accordance with the recommended therapy. Facilitating the therapy may involve outputting a recommendation for providing a statin therapy according to the statin therapy intensity to the subject. The recommendation can indicate a dosage or a range of dosages for the statin therapy based on the recommended therapy. The recommendation may additionally include information that is indicative as to why the recommendation is provided. For instance, the information may indicate which gut compositional data contributed to the recommendation.

The statin therapies, include, but are not limited to, Pitavastatin, Lovastatin, Pravastatin, Simvastatin, Atorvastatin, and Rosuvastatin. In an example, the target statin therapy may be characterized as a low intensity, a moderate intensity, or a high intensity. A low intensity may involve a daily treatment with 1 milligram (mg) of Pitavastatin, 20 mg of Lovastatin, 10 to 20 mg of Pravastatin, or 10 mg of Simvastatin. In another example, the low intensity statin therapy may involve daily treatment with 2.5 to 5 mg of Atorvastatin or 1.5 to 2.5 mg of Rosuvastatin. A moderate intensity may involve daily treatment with 2 to 4 mg of Pitavastatin, 40 to 80 mg of Lovastatin, 40 to 80 mg of Pravastatin, 20 to 40 mg of Simvastatin, 10 to 20 mg of Atorvastatin, or 5 to 10 mg of Rosuvastatin 5 to 10 mg. A high intensity may involve daily treatment with 40 to 80 mg of Atorvastatin or 20 to 40 mg of Rosuvastatin.

The therapy facilitator 130 may additionally facilitate an additional or alternative therapy based on the gut compositional data, the gut microbiome signature, and/or the output of the classifier 110. The additional or alternative therapy may include treating the subject with a composition including a cardio-metabolic probiotic, such as Akkermansia muciniphila, or a prebiotic that encourage growth of Akkermansia. As an example, the gut compositional data may indicate that the presence of Akkermansia for the subject is below a first threshold. Thus, the subject may be considered to be at a higher risk of developing side effects from a statin therapy. So, the recommended therapy may be a probiotic therapy and/or a prebiotic therapy to increase Akkermansia for the subject. The therapy facilitator 130 can output a recommendation of and facilitate the probiotic therapy and/or the prebiotic therapy for the subject. As a result, the recommendation can also include an indication of one or more additional treatments that are to be performed for the subject. In yet additional embodiments, the treating for insulin resistance includes one or more of metformin therapy, glucagon-like protein-1 (GLP-1) receptor agonist therapy, insulin therapy, cardio-metabolic probiotic therapy that can be included in the recommendation.

A communication interface 135 can collect results and communicate the result(s) (or a processed version thereof) to the provider system 125 (e.g., associated with care provider of the subject), or another system. For example, communication interface 135 may generate and output an indication of the recommended therapy. The recommendation may then be presented and/or transmitted, which may facilitate a display of the recommended therapy, for example on a display of a computing device.

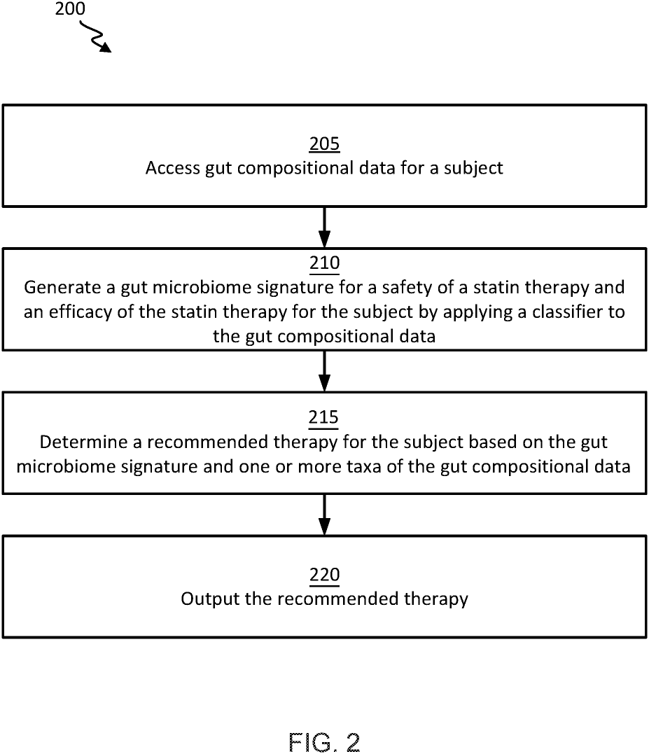

FIG. 2 illustrates an exemplary process 200 of predicting statin therapy intensity from gut compositional data according to some aspects of the present disclosure. At block 205, gut compositional data for a subject is accessed. The gut compositional data can include a taxonomic abundance of the subject, a taxonomic diversity of the subject, and/or an enterotype of the subject. The gut compositional data can be generated from fecal nucleic acid sequence data of the subject or blood metabolite data of the subject.

At block 210, a gut microbiome signature for a safety of a statin therapy for the subject and an efficacy of the statin therapy for the subject is generated by applying a classifier to the gut compositional data. The safety is characterized by an insulin resistance of the subject and the efficacy is characterized by a blood HMG level of the subject. So, the gut microbiome signature may indicate that the gut compositional data indicates a higher efficacy of the statin therapy for the subject. The classifier may be a machine-learning model trained to predict the gut microbiome signature, or the classifier may be rule-based.

At block 215, a recommended therapy for the subject is determined based on the gut microbiome signature and one or more taxa of the gut compositional data. The recommended therapy can be selected from a statin therapy intensity, a probiotic therapy, a prebiotic therapy, or a combination thereof. For instance, the recommended therapy may be a low intensity statin therapy based on the taxonomic diversity and the gut microbiome signature of the subject indicating a high efficacy. As an example, the gut compositional data may indicate a relative abundance of Bacteroides ssp. for the subject, an enterotype for the subject, and/or an alpha-diversity for the subject. The recommended therapy can be a statin therapy intensity that is inversely proportional to relative Bacteroides spp. abundance, dependent on Bacteroides enterotype assignment (e.g., whether the gut microbiome is assigned a Bacteroides enterotype or a different one such as a Ruminococacceae or Prevotella enterotype), and directly proportional to the taxonomic diversity. At block 220, the recommended therapy is output. The recommended therapy may be output to a computing device associated with a clinician of the subject such that the clinician can prescribe the recommended therapy for the subject. In addition, a dosage and statin medication for the recommended therapy may be determined based on the recommended therapy. An indication of the dosage and the statin medication can be provided to a provider system so that the appropriate statin therapy can be provided to the subject. Additional treatments, such as metformin therapy, GLP-1 receptor agonist therapy, insulin therapy, a prebiotic therapy, or cardio-metabolic probiotic therapy, may additionally be output in the recommendation for the subject.

FIG. 2 shows one exemplary process for predicting a recommended therapy from gut compositional data. Other examples can include more steps, fewer steps, different steps, or a different order of steps.

### EXAMPLES

The following examples are provided to illustrate certain particular features and/or embodiments. These examples should not be construed to limit the disclosure to the particular features or embodiments described.

### Data and Study Setting

A total of 1848 subjects were included in a cohort for a study of gut microbiome and statin therapy. The subjects were self-enrolled in a Scientific Wellness company, had available plasma metabolomics and clinical laboratory data, and provided detailed information on prescription medication use. Of the 1848 subjects, 244 identified as statin users, of which 97 provided detailed information on both dosage and type of statin prescribed. In addition, the main findings were validated in a subset of an independent European cohort (n=688), consisting of subjects at various stages of cardiometabolic disease progression, which collected stool shotgun metagenomics sequencing for gut microbiome analyses with paired medication use data, clinical laboratory test data, and serum metabolomics.

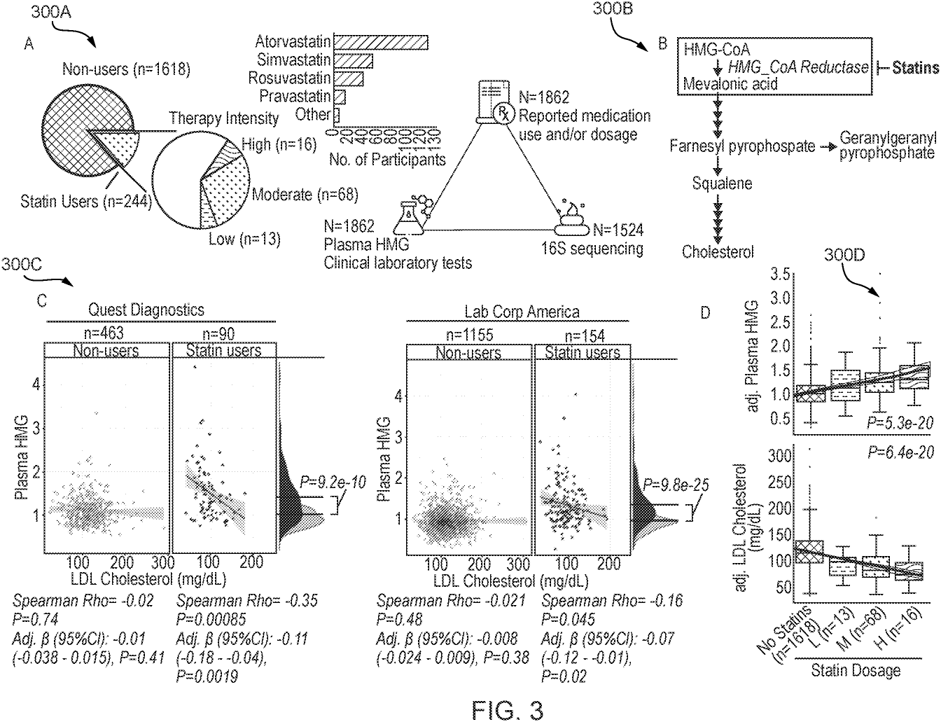

Graph 300A in FIG. 3 illustrates the frequency of statin use, type of statin taken, and number of subjects with available data for each omics from the 1864 subjects included in the study. Diagram 300B depicts de novo cholesterol synthesis, where the rate-limiting enzyme inhibited by statins is highlighted. Graph 300C depicts scatterplots of LDL-cholesterol and plasma HMG in statin non-users and users separately, across two different clinical laboratory vendors used in the cohort. The lines shown are the y~x regression lines, and the shaded regions are 95% confidence intervals for the slope of each line. Below each scatter plot is the Spearman correlation coefficient and corresponding p-value for the association between plasma HMG and LDL cholesterol. Adj. β(95%CI) corresponds to the β-coefficient (95% Confidence Interval) for LDL cholesterol from generalized linear models (GLMs) predicting plasma HMG, adjusted for sex, age, and BMI. Also shown to the right of each scatter plot are kernel density plots for plasma HMG in statin users and non-users. The lines indicate the mean of each group, and the P-value corresponds to the effect size of the difference between statin users and non-users from GLMs adjusted for the same covariates as above. Graph 300D shows the relationship between statin therapy intensity and plasma HMG as well LDL cholesterol levels for the subset of subjects in the cohort who had available dosage intensity data (n=97). The lines shown are the y~x regression lines where statin dosage intensity is coded as an ordinal variable (0(none), 1(low), 2(moderate), 3(high)), and the shaded regions are 95% confidence intervals for the slope of each line. P-value corresponds to the dose-response relationship between therapy intensity and either plasma HMG (top box plot) or LDL cholesterol (bottom box plot) (HMG: GLM adjusted for sex, age, BMI, and LDL cholesterol; LDL: ordinary least squares (OLS) regression model adjusted for sex, age, BMI and clinical lab vendor). Values on the y-axis are analyte levels adjusted for covariates (residuals). Box plots represent the interquartile range (25th to 75th percentile, IQR), with the middle line demarking the median; whiskers span 1.5 × IQR, points beyond this range are shown individually.

More specifically, the subjects consisted of adults (18+ years old) who self-enrolled in a lifestyle intervention program. The lifestyle intervention was designed to improve a number of key outcomes based on longitudinal profiling of clinical biomarkers and individualized coaching by registered nurses and dietitians. For the present study, only individuals who filled out medication questionnaires, and/or reported their prescription medication information directly, were included. Subjects further had to have available fasting plasma metabolomics and clinical laboratory test data (N=1848). Only baseline measurements and corresponding medication doses at the start of the program were considered before any lifestyle interventions were recommended. Of the 1848 subjects originally included, after excluding subjects who reported taking antibiotics in that last 3 months, 1512 had available stool 16S rRNA gene sequencing data. The majority of the subjects of this study were residents of Washington and California when in the program. Although the subjects of the cohort tend to be healthier than the general U.S. population (prevalence of obesity is 31% relative to the national prevalence of 42%), the cohort was representative of the populations in the states where the majority of the subjects were located. The cohort was further predominantly female (63%) and was skewed towards Caucasians (81%). Additional demographic information on the cohort is provided in Table 1 below. In Table 1, the number of missing values corresponds to the total number of missing values across the cohort due to either subjects not providing that information (e.g., diabetes status, race) or not having that omics data available (e.g., microbiome). ‘P-Value’ corresponds to statistical analysis testing the difference between statin users and non-users, with the type of statistical test used shown in the last column.

TABLE 1

Subject demographics stratified by statin use.

| | No. of missing values | Non-users (n=1620) | Statin users (n=244) | Whole cohort (n=1864) | P-Value | Statistical Test |
|---|---|---|---|---|---|---|
| Mean Age (s.d.) | 0 | 47.3 (10.9) | 59.1 (10.1) | 48.8 (11.5) | <0.001 | Two Sample T-test |
| Mean BMI (s.d.) | 0 | 27.8 (6.5) | 30.1 (6.2) | 28.1 (6.5) | <0.001 | Two Sample T-test |
| Mean LDL (mg/dL) (s.d.) | 0 | 115.9 (32.8) | 95.0 (28.8) | 113.2 (33.1) | <0.001 | Two Sample T-test |
| Median HOMA-IR (index) [IQR] | 0 | 1.8 [1.3,2.8] | 3.1 [2.0,5.1] | 1.9 [1.3,3.1] | <0.001 | Kruskal-Wallis |
| Mean Glucose (mg/dL) (s.d.) | 0 | 92.9 (16.5) | 106.7 (35.9) | 94.7 (20.7) | <0.001 | Two Sample T-test |
| Diabetes (n)(%) | 157 | 26 (1.8) | 40 (18.7) | 66 (3.9) | <0.001 | Chi-squared |
| Sex (n) (% Female) | 0 | 1046 (65.2) | 119 (48.8) | 1165 (63.0) | <0.001 | Chi-squared |
| Clinical lab vendor | | | | | | |
| (n) (% Quest) | 0 | 463 (28.9) | 90 (36.9) | 553 (29.9) | 0.013 | Chi-squared |
| Microbiome vendor (n) (% DNAGenotek) | 110 | 689 (45.8) | 112(48.1) | 801 (46.1) | 0.56 | Chi-squared |

Abbreviations: BMI: body mass index; LDL: low-density lipoprotein cholesterol; HOMA-IR: Homeostatic Model Assessment for Insulin Resistance; IQR: interquartile range.

Primary findings from the study were further validated in a European cohort which included 1241 subjects across the spectrum of cardiometabolic disease progression. This cohort is referred to as the MetaCardis cohort. Briefly, the MetaCardis project recruited adults from Denmark, France and Germany with increasing stages of ischemic heart disease (IHD), including 275 healthy controls (HC) matched based on demographics, 222 untreated metabolically matched controls (UMMC), 372 metabolically matched controls (MMC) and 372 subjects with IHD. Most of the subjects in the study had paired medication history, stool shotgun metagenomics sequencing data, serum metabolomics, and a subset of clinical laboratory tests. Because the overwhelming majority of IHD subjects reported taking statins or other lipid lowering drugs (~87%), the results were validated specifically in the combined HC, MMC, and UMMC groups (N=688), excluding IHD subjects, to discern the primary statin-microbiome interactions of interest from other potential drug interactions and demographic/lifestyle factors that are enriched in IHD subjects and cannot be easily adjusted for in statistical models. Further validation was also performed using strictly MMC and UMMC groups, where subjects were matched based on sex, age, BMI, and metabolic syndrome features to IHD subjects, with UMMC being further not treated with any lipid lowering medication.

### Microbiome Analysis

Stool samples were collected for each subject in the cohort using kits developed by two microbiome vendors (DNAGenotek or Second Genome). Stool sample collection kits with chemical DNA stabilizers to maintain DNA integrity at ambient temperatures were shipped directly to subjects’ homes and then shipped back to the vendors. Gut microbiome sequencing data in the form of FASTQ files were then obtained from the vendors on the basis of either the 300-bp paired-end MiSeq profiling of the 16S V3 + V4 region (DNAGenotek) or 250-bp paired-end MiSeq profiling of the 16S V4 region (Second Genome). Downstream analysis was performed using a denoise workflow that wraps functions from DADA2. DADA2 error models were first trained separately for each sequencing run and subsequently used to obtain amplicon sequence variants (ASVs) for each sample. Next, chimera removal was performed using the de novo DADA2 algorithm, which removed ~17% of all reads. Taxonomy assignment was performed using the RDP classifier with the SILVA database (version 132). In summary, 99% of the reads could be classified to the family level, 89% to the genus level and 32% to the species level. Sequence variants were aligned to each other using DECIPHER and multiple sequence alignment was trimmed by removing each position that consisted of more than 50% gaps. The resulting core alignment was then used to reconstruct a phylogenetic tree using FastTree. Gut microbiome samples were first rarefied to an even sampling depth of 25596 reads, corresponding to the minimum number of reads per sample in the dataset. Bray-Curtis and Weighted UniFrac dissimilarity matrices were calculated at the genus-level. Alpha-diversity measures were calculated at the ASV-level. Enterotype analysis was performed using Dirichlet Multinomial Mixture (DMM) modeling on the rarefied genus-level count data, which utilizes a combination of dirichlet multinomial mixtures and expectation maximization. For selecting the optimal number of DMM groups (e.g., enterotypes) in the cohort, the Bayesian information criterion (BIC) was used.

### Clinical Laboratory Tests

Blood draws for all assays were performed by trained phlebotomists at LabCorp (n=1309) or Quest (n=553) service centers, and assaying was performed in Clinical Laboratory Improvement Amendments (CLIA) certified laboratory facilities. Blood samples for clinical laboratory tests were obtained at the same time as the metabolomics blood draw. Prior to the blood draw, the subjects were advised to avoid alcohol, vigorous exercise, aspartame and monosodium glutamate for 24 hours, and to begin fasting 12 hours in advance.

### Plasma Metabolomics

Plasma HMG was measured as part of the metabolomics data generated from the same blood draws as the clinical laboratory tests. Briefly, EDTA-plasma samples were thawed on ice, after which a recovery standard was added to each sample for quality control. Aqueous methanol extraction was performed to remove the protein fraction while retaining the maximum amount of small molecular weight compounds in the sample. Sample extract was next aliquoted into five separate fractions, one for each of the four methods used for metabolite quantification, as well as one aliquot as a potential backup. Excess organic solvent was removed from the aliquoted samples by placing the samples on a TurboVap® (Zymark). Aliquoted sample extracts were stored overnight under nitrogen before analysis. All samples were run on the Waters ACQUITY ultra-performance liquid chromatography (UPLC) and a Thermo Scientific Q-Exactive high resolution/accurate mass spectrometer interfaced with a heated electrospray ionization (HESI-II) source and Orbitrap mass analyzer operated at 35,000 mass resolution. The four aliquoted sample extracts were dried then reconstituted in solvents compatible with each of the four methods used for downstream metabolite quantification. To ensure injection and chromatographic consistency, each solvent further contained a series of standards at fixed concentrations. Two of the four aliquots were analyzed using acidic positive ion conditions chromatographically optimized for either more hydrophobic (solvent consisting of water, methanol, acetonitrile, 0.05% perfluoropentanoic acid (PFPA) and 0.01% formic acid (FA)) or hydrophilic compounds (water and methanol, containing 0.05% PFPA and 0.1% FA). Both of these aliquots were eluted using a C18 column (Waters UPLC BEH C18-2.1 × 100 mm, 1.7 µm). Elution for aliquot 3 was performed using a dedicated C18 column in solvent containing methanol and water under basic negative ion optimized conditions, with 6.5 mM Ammonium Bicarbonate at pH 8. The fourth and final aliquot was analyzed via negative ionization following elution from a HILIC column (Waters UPLC BEH Amide 2.1 × 150 mm, 1.7 µm) using a gradient consisting of water and acetonitrile with 10 mM Ammonium Formate, pH 10.8. Mass spectrometry (MS) analysis was performed using dynamic exclusion and alternating between MS and data-dependent MSn scans. The scan range varied slightly between the four methods used, and covered 70-1000 m/z. Process blanks and EDTA-plasma technical replicates were run intermittently throughout the study run-days to account for potential run and day variability. A biochemical library of over 3300 purified standards based on chromatographic properties and mass spectra was used for identification of known chemical entities. Raw metabolomics data was next normalized as described previously. Values were median scaled within each batch, such that the median value for each metabolite was 1. To adjust for possible batch effects, further normalization across batches was performed by dividing the median-scaled value of each metabolite by the corresponding average value for the same metabolite in technical control samples processed in the same batch. The same technical control samples were used to ensure the comparability of abundance estimates obtained across batches.

### Genetic Analysis

Subject DNA was extracted from whole blood and, following quality control and purification, as needed, underwent 150 paired-end (PE) whole genome sequencing (WGS) using Illumina’s HiSeq X at 30x coverage. Variant calling was performed using the pipeline that follows Genome Analysis Toolkit’s (GATK’s) Best Practices, using Haplotype Caller and hg19 build as the reference genome. A total of 1747 subjects (~94% of the present cohort) had available WGS data and were used in the analysis. Following quality control and assurance, genetic ancestry was calculated as principal components (PCs) using a set of ~100,000 ancestry-informative SNP markers as described previously. SNPs chosen for testing associations with HMG were based on prior studies investigating genetic predisposition to statin efficacy defined as percent decrease in LDL-cholesterol from baseline, and included the following variants: rs10455872, rs2199936, rs2900478, rs4420638, rs445925, rs5908, rs646776, rs7412, and rs8014194. To model the association between SNPs and HMG in statin users, subjects homozygous and heterozygous for the minor allele were grouped together. Statistical analysis was performed on each SNP individually using generalized linear models (GLM) with a Gamma distribution and a log-link function, with HMG as the dependent variable and a statin-by-SNP interaction term. The interaction term tested for a significant association between HMG and statin use, that was modified by the SNP of interest (e.g., the effect of statins on HMG are variable based on the genetic variant). Models were further adjusted for sex, age, BMI and the first 7 ancestry PCs. Ordinary Least Square (OLS) regression models with the same covariates and interaction term were also run with LDL-cholesterol as the dependent variable. Type-1 error was controlled using the Benjamini-Hochberg method (FDR<0.05).

### Statistical Analysis

Of the 1848 subjects included in the study, 73 had missing data on sex and age, 66 on BMI, 81 on HMG and 6 on LDL-cholesterol. These missing values were imputed using plasma metabolomics data and a K nearest neighbor algorithm. The associations of plasma HMG levels with LDL-cholesterol, statin intensity, and measures of gut alpha-diversity were all tested using GLM with a Gamma distribution and a log-link function, with HMG as the dependent variable. OLS regression was used when LDL-cholesterol or measures of gut alpha-diversity were the dependent variables. Testing for associations between variables and interindividual variability in gut microbiome composition was conducted using permutational multivariate analysis of variance (PERMANOVA) using both the genus-level Bray-Curtis and Weighted UniFrac dissimilarity matrices. The number of permutations to obtain P-values was set to 3000.

For assessing dose-response relationships between HMG/LDL-cholesterol and dosage intensity (FIG. 3D), dosage was recoded into an ordinal variable (0(none/no statins), 1(low), 2(moderate), 3(high)), and the significance of the β-coefficient for that variable from covariate adjusted models predicting either HMG (GLM adjusted for sex, age, and BMI) or LDL-cholesterol (OLS adjusted for sex, age, BMI, and clinical lab vendor) was reported. Wherever associations were visualized using box plots or scatter plots, the residuals (values adjusted for covariates from either GLM or OLS models) were plotted instead of the original values. For comparing the differences in prevalence of the four enterotypes among statin users and non-users, the χ² test was performed. When evaluating the association between obesity and Bac.2 enterotype, as well as statin use and Bac.2 enterotype among obese subjects, multivariable logistic regression models were generated with Bac.2 membership (versus all other enterotypes) as the dependent variable.

When testing for significant enterotype-by-statin interactions, HMG and metabolic parameters (blood glucose, blood insulin, HOMA-IR, and HbA1c) were log transformed prior to fitting the models. Analysis of Variance (ANOVA) or covariance (ANCOVA) models were then used to test for significant interactions (ANOVA (measure ~ statin_use+enterotype+statin_use*enterotype) for unadjusted models and ANCOVA (measure~covariate 1+covariate 2+...covariate X+statin_use+enterotype+statin_use*enterotype) for covariate adjusted models). If a significant interaction was present, post-hoc comparisons were performed between statin users and non-users within each enterotype on the covariate adjusted values (residuals) using two-sample t-tests, with Bonferroni corrected P<0.05 considered statistically significant.

### Relationship of Plasma HMG, Statin Use, and On-Target Effects

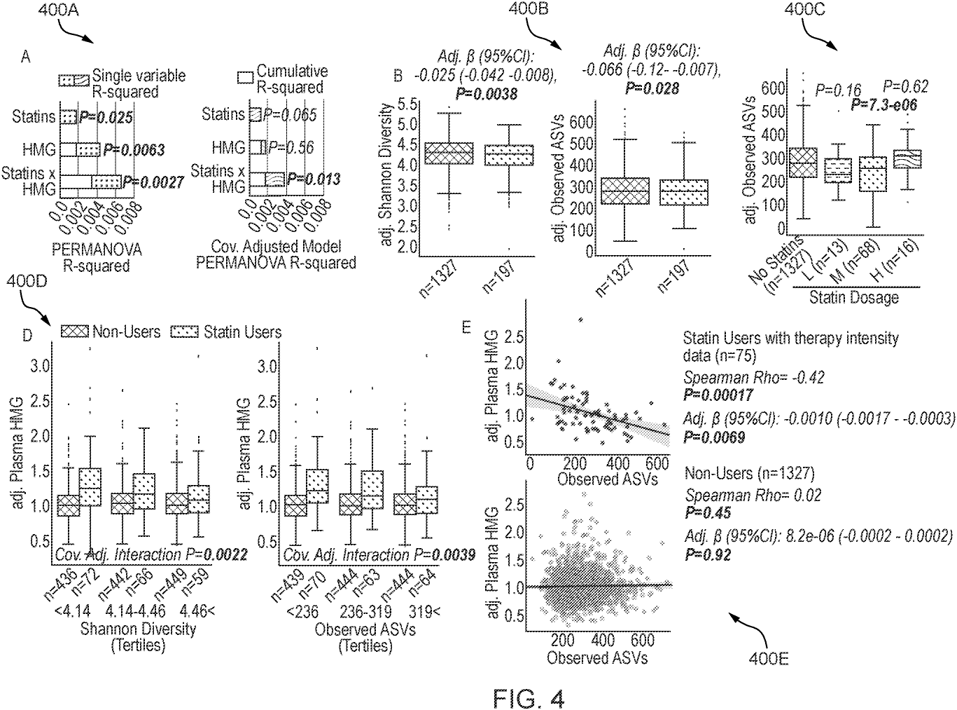

The mechanism of action of statins is to inhibit the rate-limiting enzyme of de novo cholesterol synthesis, 3-hydroxy-3-methylglutaryl coenzyme A (HMG-CoA) reductase. Thus, the study sought to evaluate whether elevated plasma levels of the hydrolyzed substrate for the enzyme, HMG (measured in the broad untargeted metabolomics panel), could serve as a reliable marker of statin use (FIG. 4B). Plasma HMG levels were significantly higher in statin users than in non-users, consistent with the initial hypothesis and the mechanism of action of statins (FIG. 4C, generalized linear models (GLMs) adjusted for sex, age, and BMI, Quest Diagnostics β(95% confidence interval (CI)): 0.23 (0.16-0.31), P=9.2e-10), Lab Corp. of America (LCA) β(95% CI):0.28(0.23-0.34), P=9.8e-25). HMG levels further showed a negative correlation with blood LDL-cholesterol across two independent entities, but exclusively in statin users, indicating that plasma HMG may not only reflect statin use but also the extent to which statins inhibit their target enzyme (FIG. 4C, GLM adjusted for sex, age, and BMI, Quest Diagnostics β(95% CI): -0.12 (-0.19-0.05), P=0.0016), LCA β(95% CI):-0.07(-1.2 - -0.01), P=0.020)).

To further evaluate the robustness of HMG as a marker for statin on-target effects, the correspondence of HMG to variable doses of statins prescribed in a subset of statin users where this information was available (n=97) was explored. Different statins (atorvastatin, simvastatin, etc.) exhibit different potencies and are often prescribed at variable doses. In order to synchronize medical practices in terms of statin therapy, the American Heart Association (AHA) released guidelines for adjusting statin doses across all types of statins, which cluster into one of three intensity categories (low, moderate, and high) aimed at achieving desired decreases in LDL-cholesterol of <30%, 30-49%, ≥50%, respectively. Based on these AHA guidelines, a daily 40 mg dose of Rosuvastatin would place a subject in the high intensity category, while the same dose of Fluvastatin would place a subject in the low intensity group. Hence, the subjects were reclassified into their respective therapy intensity groups based on the AHA guidelines (FIG. 4A) and evaluated the associations between therapy intensity, plasma HMG, and blood LDL-cholesterol levels. Therapy intensity showed a positive dose response relationship with HMG, independent of sex, age, and BMI (adj. β(95% CI):0.15(0.12-0.17), P=1.1e-22)). Consistently, an inverse relationship was observed between therapy intensity and blood LDL-cholesterol (FIG. 4D, β(95% CI):-15(-18 - -12), P=6.7e-20, adjusted for sex, age, BMI and clinical lab vendor)).

Referring to FIG. 4, gut microbiome is shown to modify statin efficacy. Graph 400A shows the proportion of variance explained by statin use, plasma HMG levels, and a statin-by-HMG interaction term from unadjusted PERMANOVA models (statin use + HMG + statin use x HMG) or models adjusted for sex, age, BMI, microbiome vendor, and LDL cholesterol using the Weighted UniFrac genus-level dissimilarity matrix. Grey area corresponds to the cumulative R-squared of variables added to the model prior to the variable indicated on the x-axis, while the other areas of the bars represent the additional variance explained by that variable. Graph 400B show measures of gut alpha-diversity in statin users compared to non-users. The Beta-coefficient, 95%CI and P-value shown for each of the boxplots is derived from OLS models predicting each of the log(alpha-diversity) measures adjusted for microbiome vendor, sex, age, BMI, and LDL cholesterol. Values on the y-axis are diversity measures adjusted for these covariates (residuals). Graph 400C shows measures of observed ASVs in statin users and non-users with known statin therapy intensity (low, moderate, high). P-values shown correspond to beta-coefficients from OLS models predicting log(observed ASVs) comparing each intensity group to the no statin control group, adjusted for the same covariates as in graph 400B. Values on the y-axis are diversity measures adjusted for these covariates. Graph 400D depicts plasma HMG levels among statin users and non-users across tertiles of gut-alpha diversity. Interaction P corresponds to the statin*alpha diversity measure interaction term P-value from GLM predicting plasma HMG adjusted for the same covariates as in graphs 400B-C. Values on the y-axis are diversity measures adjusted for these covariates. Graph 400E shows scatterplots of observed ASVs (x-axis) and covariate adjusted plasma HMG levels (y-axis) in statin users with known dosage therapy intensity as well as statin non-users. Also provided are the spearman correlation coefficients and their corresponding P-value, as well as adjusted B-coefficients from GLMs predicting HMG levels adjusted for the same covariates as in graphs B-D, as well as statin intensity. For all box plots shown, box plots represent the interquartile range (25th to 75th percentile, IQR), with the middle line denoting the median; whiskers span 1.5 × IQR, points beyond this range are shown individually.

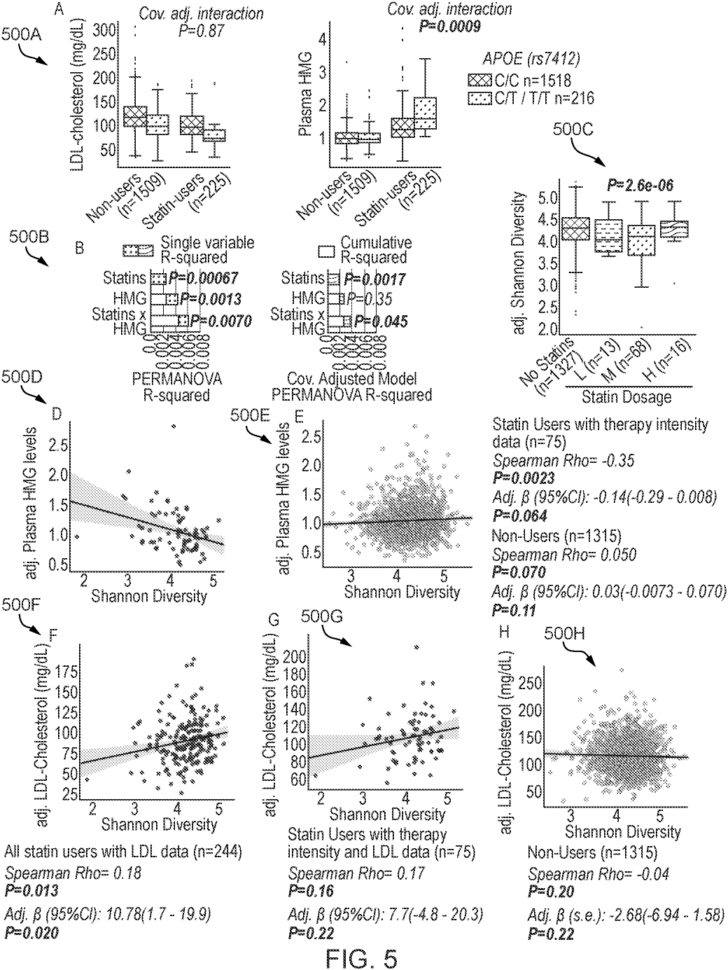

To evaluate if plasma HMG captures known genetic variability in statin response, the associations between HMG and 9 SNPs most strongly associated with statin-mediated decrease in LDL-cholesterol were tested, using GLMs with a statin-by-genetic variant interaction term while adjusting for sex, age, BMI and genetic ancestry. Of the 9 SNPs tested, 2 SNPs in close linkage disequilibrium (rs445925 and rs7412 mapping to the APOC1 and APOE genes, respectively, r > 0.80 in Caucasians) showed significant associations with HMG, that were dependent on statin intake (e.g., the effect was only present in statin users, FDR<0.05), in the directions consistent with the previously described associations of the same variants with statin response (FIG. 5, Table 2). Running the same analysis with LDL-cholesterol instead of plasma HMG as an outcome variable (both measured from the same blood draw) did not reveal the same statin-dependent interactions (Table 2). In the case of both rs445925 and rs7412, carrying at least one copy of the minor allele was associated with a decrease in LDL-cholesterol across statin users and non-users alike, hence providing no additional insight into statin-mediated effects (FIG. 5). Together, the combined analyses of statin use, statin therapy intensity and genetic variants known to modify statin response indicate that HMG may provide additional insight into statin on-target effects, not captured by a snapshot measurement of LDL-cholesterol in a cross-sectional study.

### Relationship of Statin Use and Gut Microbiome

Given the associations between the gut microbiome and statin use, the next investigation evaluated whether statin intake is associated with changes in gut microbiome composition. Statin use showed a significant association with interindividual variability in gut microbiome composition, using the Bray-Curtis dissimilarity metric (PERMANOVA unadjusted model R2=0.0025, P=0.00067, model adjusted for microbiome vendor, sex, age, and BMI, R2=0.0021, P=0.0017) and Weighted UniFrac (unadjusted model R2=0.0017, P=0.031, model adjusted for the same covariates as the Bray-Curtis model, R2=0.0013, P=0.065) (FIG. 4A, FIG. 5). Association between statin use and measures of gut α-diversity were further tested by calculating observed Amplicon Sequence Variants (ASVs), a measure of species richness reflecting the number of unique taxa in the ecosystem, and Shannon diversity, a correlated measure that captures both richness and evenness in the abundances of taxa present. Statin intake was further associated with a significant modest decrease in one of the two alpha-diversity metrics calculated (OLS regression predicting Shannon diversity adjusted for the same covariates as PERMANOVA, adj. β(95% CI):-0.095 (-0.16 - -0.028), P=0.0051) (FIG. 4B). When looking at specific statin therapy intensity for a subset of subjects where this information was available, there was no monotonic dose-response relationship between gut alpha-diversity, with only subjects receiving moderate intensity statin therapy demonstrating a significant decrease in measures of gut alpha-diversity relative to non-users (FIG. 4C, FIG. 5).

Referring to FIG. 5, gut alpha-diversity is shown to be anti-correlated with statin on-target effects. Graph 500A shows LDL-cholesterol and plasma HMG measures in subjects stratified by statin use and genotype. Provided is the P-value for the statin-by-SNP interaction term from GLM (HMG) or OLS (LDL) models adjusted for sex, age, BMI and the first 7 ancestry principle components. Graph 500B shows the proportion of variance explained by statin use, plasma HMG levels, and a statin-by-HMG interaction term from unadjusted PERMANOVA models (statin use + HMG + statin use-by-HMG) or models adjusted for sex, age, BMI, and microbiome vendor using the Bray-Curtis genus-level dissimilarity matrix. The grey area corresponds to the cumulative R-squared of variables added to the model prior to the variable indicated on the x-axis, while the other areas of the bars represent the additional variance explained by that variable. Graph 500C shows measures of observed ASVs in non-users and across statin users with known therapy intensity (low, moderate, high). Graphs 500D-E depict scatterplots of Shannon diversity (x-axis) and covariate adjusted plasma HMG levels (y-axis) in statin users with known dosage therapy intensity (graph 500D) and statin non-users (graph 500E). HMG values have been adjusted for the same covariates as in graph 500B, as well as statin intensity. Also provided are the spearman correlation coefficients and their corresponding P-value, as well as adjusted β-coefficients from GLM predicting HMG levels adjusted for the same covariates as in graph 500C) as well as dosage intensity. Graphs 500F-G are scatterplots of Shannon diversity (x-axis) and covariate adjusted LDL-cholesterol levels (y-axis) in all statin users (graph 500F) and statin users with known therapy intensity (graph 500G), where LDL values were further adjusted for therapy intensity. Graph 500H shows a scatterplot of Shannon diversity (x-axis) and covariate adjusted LDL-cholesterol (y-axis) in statin non-users adjusted for the same covariates as in graph 500F).

### Relationship of Gut Microbiome and Statin Efficacy

Next, an association between gut microbiome beta-diversity and interindividual heterogeneity in response to statin therapy was evaluated. Using HMG as a proxy for statin inhibition of its target enzyme, the correspondence between statin on-target effects and interindividual variability in gut microbiome beta-diversity was modeled using PERMANOVA and including a statin-by-HMG interaction term. The interaction terms had permutation-based p-values of 0.0070 (R2=0.0017) and 0.0013 (R2=0.0032) for Bray-Curtis and Weighted Unifrac metrics, respectively, which remained significant after adjusting for microbiome vendor, BMI, sex, and age (Bray-Curtis R2=0.0011, P=0.045, W. Unifrac R2=0.0020, P=0.012) (FIG. 4A, FIG. 5). These results indicate that HMG correspondence to gut microbiome composition is dependent on statin intake, similar to the HMG-SNP associations reported earlier (FIG. 5). Very similar patterns were observed for gut alpha-diversity, where, once again, the association between the proxy for statin efficacy, HMG, and gut alpha-diversity was dependent on statin intake (FIG. 4D, GLMs adjusted for microbiome vendor, sex, age, and BMI, Shannon diversity-by-statin interaction term β(95% CI):-0.15(-0.25 - -0.060), P=0.0014, Observed Amplicon Sequence Variants ASVs in a sample (observed ASVs)-by-statin interaction term β(95% CI):-0.00060(-0.001 - -0.0002), P=0.0033). Plotting the association between gut alpha-diversity and HMG stratified by statin use revealed that, among statin users, higher alpha-diversity corresponded to lower plasma HMG levels, indicating decreased on-target effects of the therapy in subjects with more diverse microbiomes (FIG. 4D). The negative association between HMG and alpha-diversity in statin users was also orthogonal to genetic variants predisposing subjects to variable statin responses. Running a stepwise forward regression model predicting log transformed HMG levels using the 9 SNPs previously associated with statin response explained an additional 3.2% of variance in HMG, on top of age (e.g., the base model). Including observed ASVs as a measure of gut diversity in the model in addition to age and the chosen SNPs increased the percent variance explained by an additional 3.9% (complete model R2=0.185).

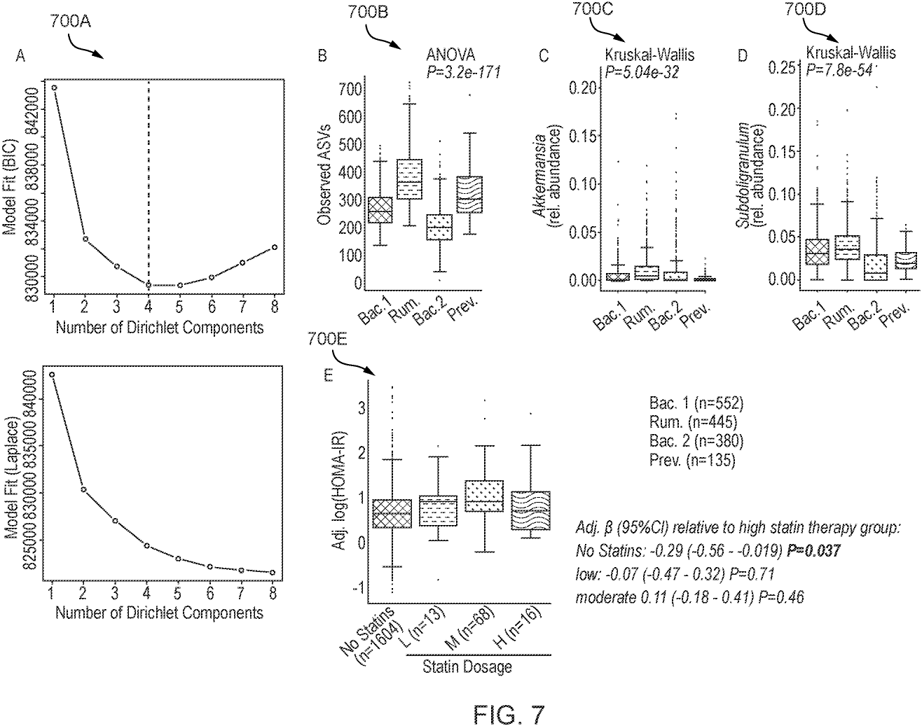

To further exclude the possibility that subjects with higher alpha-diversity are generally healthier and simply prescribed less potent statin therapies to begin with, thus leading to lower levels of HMG, the models were further adjusted for dosage intensity in the subset of subjects with gut microbiome compositional data where this information was available (n=75). In the smaller group of subjects, associations between gut alpha-diversity and HMG were not impacted by correcting for statin intensity (FIG. 4E & FIG. 7). Similar results were observed when investigating statin dependent associations between LDL-cholesterol and gut alpha-diversity, although to a weaker extent (OLS models predicting LDL-cholesterol adjusted for clinical lab and microbiome vendors, sex, age, and BMI, statin-by-Shannon diversity interaction term β(95% CI): 12.2(2.5-22.0), P=0.014, statin-by-Observed ASVs interaction term β(95% CI):0.042(0.00086-0.084), P=0.044, FIG. 7). A weaker interaction effect with LDL cholesterol was expected, given the cross-sectional nature of the study and the inability to capture the percent decrease in LDL-cholesterol from baseline following the initiation of statin treatment, one of the most common and direct measures of statin effectiveness.

As another measure of gut microbiome correspondence to statin response, the association between measures of gut alpha-diversity and the likelihood of having reached predefined target LDL-cholesterol levels for statin users (<70 mg/dL and <100 mg/dL) was evaluated. These are clinically relevant targets, as clinicians are recommended to adjust dosage and type of statin prescribed to reach these particular levels of LDL-cholesterol depending on the presence of specific ASCVD risk factors in their subjects. Both Shannon diversity and Observed ASVs showed negative associations with likelihood of having reached target LDL-levels among statin users (Multivariable logistic regression adjusted for clinical lab vendor, sex, age, BMI, and T2D status (a common criteria, in combination with one or more CVD risk factors, where more aggressive LDL-lowering therapy is pursued): Odds Ratios (OR) ranging from 0.60-0.69, Table 3). Together, these results indicate that gut microbiome composition can explain a significant proportion of variability in statin on-target effects in a generally healthy community-dwelling population.

### Relationship Between Gut Compositional Data and Statin Efficacy and Glucose Homeostasis

Statin intake among obese subjects is associated with lower prevalence of the Bacteroides 2 (Bac.2) enterotype, which is generally considered to be less healthy than other broad enterotype groupings common to cohorts in the United States and Europe. To evaluate the extent to which these coarse ecological groupings might help explain interindividual variation in statin on- and off-target effects, the subjects were stratified into enterotypes. Dirichlet multinomial mixture (DMM) modeling was used to separate the subjects into four groups, according to the Bayesian Information Criterion (BIC), consistent with some, but not all, previous human gut microbiome studies (Bacteroides 1 (Bac.1), Bac.2, Ruminococcaceae (Rum.), and Prevotella (Prev.) clusters) (FIG. 5A, FIG. 7). The four enterotypes identified showed very similar characteristics to those described previously in European cohorts, with two Bacteroides-dominated enterotypes (Bac.1 and Bac.2), with the Bac.2 enterotype being further characterized by decreased alpha-diversity and a depletion of SCFA-producing commensals like Faecalibacterium and Subdoligranulum (FIG. 5B, FIG. 7). The Rum. enterotype was enriched for taxa primarily from the Firmicutes phylum, as well as Akkermansia (FIG. 7, Table 2). The Prev. enterotype was the smallest in size and characterized by high relative abundance of the Prevotella genus (FIG. 5D, Table 2).

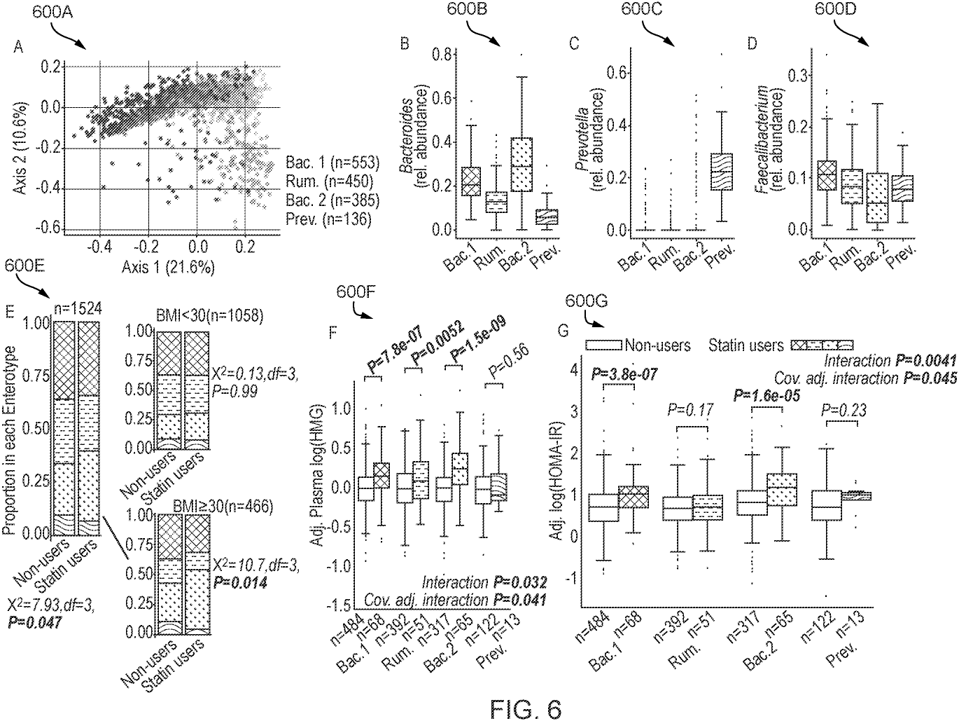

Referring to FIG. 6, microbiome enterotypes are shown to modify statin efficacy and metabolic side effects. Graph 600A is a Principle Coordinate Analysis (PCoA) plot of the genus-level Bray-Curtis Dissimilarity matrix separated by enterotypes. Graphs 600B-D depict the relative abundance of Bacteroides (graph 600B), Prevotella (graph 600C), and Faecalibacterium (graph 600D) across the four enterotypes identified in the cohort. Graph 600E shows the proportion of each enterotype in statin users and non-users across the whole cohort (left) and stratified by obesity (right). Chi-square test values, degrees of freedom and corresponding P-values are provided testing for significant difference in proportion of enterotypes between statin users and non-users across the whole cohort and stratified by obesity. Graph 600F shows plasma HMG levels among statin users and non-users stratified by enterotype. Interaction P corresponds to the statin*enterotype interaction term P-value from unadjusted ANOVA models, while the cov. Adj. interaction P corresponds to the statin*enterotype interaction term P-value from ANCOVA models adjusted for microbiome vendor, sex, age, BMI and LDL cholesterol. Plasma HMG levels shown on the y-axis are values adjusted for the same covariates. P-values above the box plots correspond to tests of significance between statin non-users and statin users within each enterotype using two-samples t-test. Differences with Bonferroni corrected P<0.05 were considered statistically significant. Graph 600G shows HOMA-IR measures among statin users and non-users stratified by enterotype. Interaction P corresponds to the statin*enterotype interaction term P-value from unadjusted ANOVA models, while the cov. Adj. interaction P corresponds to the statin*enterotype interaction term P-value from ANCOVA models adjusted for clinical lab vendor, microbiome vendor, sex, age, BMI, HMG and LDL cholesterol. HOMA-IR levels shown on the y-axis are values adjusted for the same covariates. P-values above the box plots correspond to tests of significance between statin non-users and statin users within each enterotype using two-samples t-test. Differences with Bonferroni corrected P<0.05 were considered statistically significant. Box plots represent the interquartile range (25th to 75th percentile, IQR), with the middle line denoting the median; whiskers span 1.5 × IQR, points beyond this range are shown individually.

FIG. 5 shows enterotypes differ in their relative abundance of SCFA-producing taxa. Graph 700A depicts the measure of model fit using the Bayesian information criterion (BIC) (top) across an increasing number of Dirichlet components as well as Laplace approximation (bottom) in the subjects. Specifying 4 components resulted in best model performance using BIC and is highlighted by the dotted line. Graph 700B depicts gut alpha-diversity measures using observed ASVs across the four enterotypes. Graphs 700C-D compare relative abundance of the genus Akkermansia (graph 700C) and Subdoligranulum (graph 700D) across the four enterotypes identified in the subjects. P-value from a non-parametric Kruskal-Wallis test comparing differences across all four enterotypes is provided in the top right corner. Graph 700E shows HOMA-IR levels across statin non-users and statin users with known therapy intensity. To the right are the β-coefficients, 95% confidence intervals, and P-values from OLS regression models predicting log(HOMA-IR) adjusted for clinical lab vendor, microbiome vendor, sex, age, BMI, and LDL cholesterol. HOMA-IR values on the y-axis have been adjusted for the same covariates. Box plots represent the interquartile range (25th to 75th percentile, IQR), with the middle line denoting the median; whiskers span 1.5 × IQR, points beyond this range are shown individually.

TABLE 2

Correspondence of HMG with SNPs associated with statin response

| | | HMG (N=1734) | | | | LDL-Cholesterol (N=1734) | | | |
|---|---|---|---|---|---|---|---|---|---|
| SNP rsid | At least one copy of the minor allele (proportion) | Adj. β-coeff | s.e. | P-value | Corr. P-value | Adj. β-coeff | s.e. | P-value | Corr. P-value |
| rs10455872 | 0.11 | -0.0207 | 0.0338 | 0.5402 | 0.6690 | -4.5819 | 3.5322 | 0.1947 | 0.4333 |
| rs2199936 | 0.23 | -0.0157 | 0.0245 | 0.5211 | 0.6690 | -0.2432 | 2.5747 | 0.9247 | 0.9966 |
| rs2900478 | 0.29 | -0.0110 | 0.0237 | 0.6427 | 0.6690 | 1.0850 | 2.4733 | 0.6609 | 0.9966 |
| rs4420638 | 0.30 | -0.0178 | 0.0228 | 0.4350 | 0.6690 | -3.4403 | 2.3689 | 0.1466 | 0.4333 |
| rs445925 | 0.18 | 0.0907 | 0.0324 | 0.0052 | 0.0207 | -0.0143 | 3.3645 | 0.9966 | 0.9966 |
| rs7412 | 0.12 | 0.1513 | 0.0453 | 0.0009 | 0.0068 | 0.7567 | 4.6668 | 0.8712 | 0.9966 |
| rs646776 | 0.37 | 0.0096 | 0.0224 | 0.6690 | 0.6690 | 4.1731 | 2.3359 | 0.0742 | 0.4333 |
| rs8014194 | 0.46 | 0.0347 | 0.0215 | 0.1068 | 0.2849 | 2.7833 | 2.2520 | 0.2166 | 0.4333 |

β-coefficients, standard error (s.e.) and the corresponding p-value for the SNP-by-statin interaction term predicting either HMG (GLM) or LDL-cholesterol levels (OLS regression) across the subjects with available genetics data. Models were adjusted for sex, age, BMI and the first 7 ancestry PCs. “Corr. P-value” corresponds to the P-value for each β-coefficient after correcting for multiple hypothesis testing (FDR<0.05). Significant P-values are underlined

However, BIC as a model penalization metric is not without limitations and tends to err on the side of underfitting (e.g., estimating a smaller number of clusters). The Laplace approximation for model penalization, on the other hand, did not identify an optimal number of clusters in this particular dataset (out to a maximal number of eight clusters tested), indicating limited statistical evidence for a small number of coarse-grained compositional states within the cohort (FIG. 7). Nevertheless, the main enterotype groupings tend to be relatively consistent from study-to-study in large U.S. and European populations, even if the statistical evidence for such states is somewhat limited.

Consistent with previous results, obesity itself was associated with a higher likelihood of being assigned to the Bac.2 enterotype (Multivariable logistic regression adjusted for microbiome vendor, sex, and age, OR(95%CI): 1.8 (1.4-2.3), P=5.0e-5). Additionally, the study observed a higher prevalence of the Bac.2 enterotype among statin users compared to non-users, particularly among obese subjects (FIG. 5E). This association among obese subjects was further confirmed using multivariable logistic regression adjusting for sex, age, and microbiome vendor (OR(95%CI): 2.1 (1.2-3.7), P=0.013, n=462).

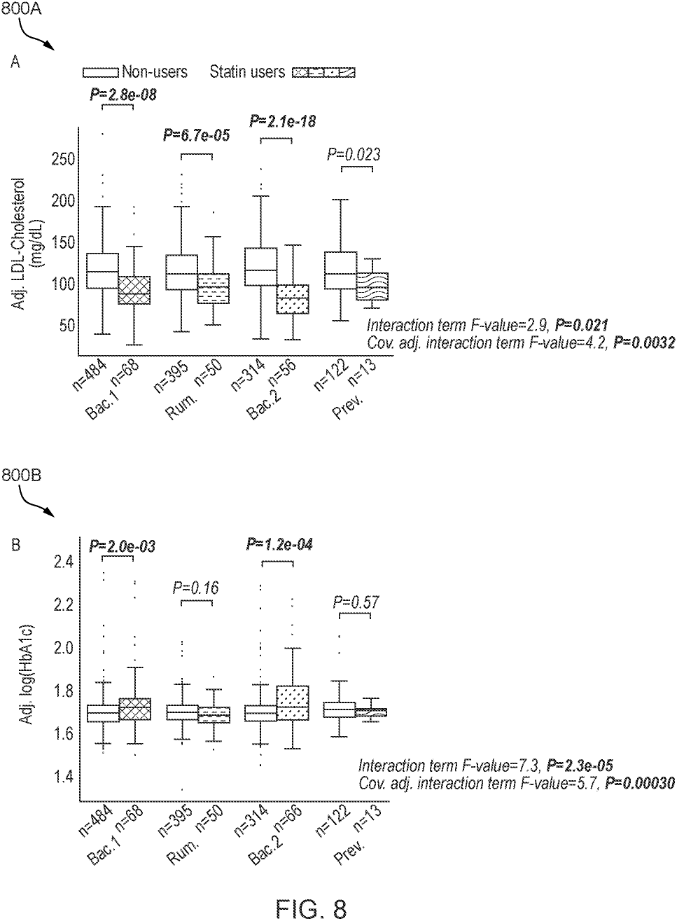

Next, an association between a subject’s enterotype with their response to statin therapy was explored. Focusing on statin on-target effects, the study observed a significant enterotype-by-statin interaction when predicting blood HMG levels (P=0.044, unadjusted analysis of variance (ANOVA), P=0.034, analysis of covariance (ANCOVA) adjusted for microbiome vendor, clinical lab vendor, sex, age, and BMI). Stratifying the cohort by enterotypes and comparing statin users to non-users revealed that the Bac.2 enterotype displayed the greatest increase in HMG with statin use (37% mean increase), followed by the Bac.1 (24%) and Rum. enterotypes (18%). Subjects with a Prev. enterotype showed no significant increase in HMG while on statins, although thesample size for this particular enterotype was small and thus this result may need to be interpreted with caution (FIG. 5F). Similar results were obtained when evaluating statin-by-enterotype interaction effects on LDL-cholesterol levels (P=0.021, unadjusted ANOVA, P=0.0032, ANCOVA adjusted for same covariates as HMG models), with the Bac.2 enterotype demonstrating the greatest mean LDL decrease (-33%) relative to non-users within the same enterotype (FIG. 8). Statin users who were assigned the Bac.2 enterotype were also two to four-times more likely to have reached common LDL-cholesterol target levels for statin-users at higher risk for ASCVD (Table 3). These results suggest that microbiome enterotypes may reflect the extent to which statins inhibit HMG-CoA reductase and reduce LDL-cholesterol levels across subjects.

TABLE 3

Gut microbiome measures correlate with having reached LDL-cholesterol target levels among statin users

| | <100 mg/dL (n cases=132, N total=197) | | <70 mg/dL (n cases=44, N total=197) | |
|---|---|---|---|---|
| | Cov. adj. OR(95%CI) | Cov. & T2D adj. OR(95%CI) | Cov. adj. OR(95%CI) | Cov. & T2D adj. OR(95%CI) |
| Shannon diversity | 0.69 (0.49-0.97) | 0.72 (0.50-1.03) | 0.67 (0.48-0.95) | 0.60 (0.41-0.87) |
| Observed ASVs | 0.67 (0.47-0.95) | 0.67 (0.45-0.98) | 0.66 (0.45-0.95) | 0.62 (0.40-0.96) |
| Bac.2 enterotype | 2.19 (1.04-4.60) | 2.11 (0.95-4.66) | 3.61 (1.68-7.77) | 4.33 (1.83-10.25) |

Odds Ratios (OR) for each gut microbiome measure from logistic regression models predicting having achieved either <100 mg/dL or <70 mg/dL target LDL-cholesterol level among statin users. The Bac.2 enterotype was compared against all other enterotypes. Measures of alpha-diversity were scaled and centered prior to analysis for easier comparison of effect sizes. Models were adjusted for clinical laboratory and microbiome vendors, age, sex and BMI. Further adjustment for T2D status was done in participants where this information was available (n=1691). Significant OR (P<0.05) are underlined.

### Prediction of Statin Side Effects by Gut Microbiome Composition

Statin use has previously been associated with disrupted glucose control and increased risk of developing T2D in a subset of subjects. Given the known role of the gut microbiome in contributing to metabolic homeostasis, and the variable metabolic profiles previously observed across different microbiome enterotypes, the study investigated whether enterotypes may modify the association between statin use and markers of insulin resistance. Focusing initially on Homeostatic Model Assessment for Insulin Resistance (HOMA-IR), the study tested for an enterotype-by-statin interaction effect while adjusting for microbiome vendor, clinical lab vendor, sex, age, BMI, LDL-cholesterol, and plasma HMG using ANCOVA. Subjects showed variable responses to statin therapy based on their microbiome enterotype, with Bac.2 subjects on statins demonstrating the highest increase in HOMA-IR relative to non-statin users, while Rum. subjects showed no significant increase in HOMA-IR between statin users and non-users (ANOVA unadjusted interaction term P=0.0037, ANCOVA covariate adjusted Interaction term P=0.0495, FIG. 5G, Table 4). In the subset of subjects where dosage intensity information was available, all three intensities (low, moderate, high) were associated with a comparable increase in HOMA-IR, suggesting that differences in therapy intensity are likely not the main driver behind the observed statin-enterotype interaction (FIG. 7).

The study then expanded the analysis into additional markers of metabolic health, including fasting insulin and blood glucose, as well as glycated hemoglobin A1c. There was a significant enterotype-by-statin interaction across all tested metabolic parameters, which remained significant after adjusting for covariates across all markers other than insulin (Table 4, FIG. 8). As subjects with T2D are often recommended to take statins, the study further adjusted all models for T2D status in subjects where this information was available (N=1691, T2D n=66), which did not change the significance of enterotype-by-statin interaction effects observed (Table 4). Because a subset of subjects on statins is often concurrently treated with glucose- controlling medication, the ANCOVA models were further adjusted for metformin use (the most commonly reported glucose-controlling drug in the cohort), which did not drastically change the significance of the enterotype-by-statin interaction effects observed. Collectively, these results suggest that gut microbiome composition may modify how statins influence off-target physiology, particularly glucose homeostasis.

TABLE 4

Gut microbiome enterotypes modify the association between statin use and markers of glucose homeostasis

| Measure | Percent median increase in each measure and P-value | | | | F-value and corresponding P-value for statin*enterotype interaction term predicting each measure | | |
|---|---|---|---|---|---|---|---|
| | Between statin-users and non-users for each enterotype | | | | | | |
| | Bac.1 | Rum. | Bac.2 | Prev. | Unadjusted model N=1848 | Covariate adj. model N=1848 | Covariate and diabetes adj. model N=1691 |
| HOMA-IR | 73%, P=7.2e- 07 | 21% P=0.27 | 99% P=1.2e- 04 | 29% P=0.33 | F=4.5, P=0.0037 | F=2.6, P=0.0495 | F=2.6, P=0.049 |
| Insulin | 63% P=5.6e- 06 | 19% P=0.17 | 89% P=9.1e- 04 | 22% P=0.25 | F=3.0, P=0.032 | F=1.4, P=0.23 | F=1.5, P=0.22 |
| Glucose | 6.6% P=9.7e- 04 | 4.5% P=0.51 | 9.3% P=8.1e- 04 | 7.6% P=0.84 | F=6.4, P=0.00025 | F=4.4, P=0.0041 | F=3.9, P=0.0092 |
| HbAlc | 5.6% P=2.0e- 03 | 1.9% P=0.16 | 7.3% P=1.2e- 04 | 1.8% P=0.57 | F=8.1, P=2.3E-05 | F=6.3, P=0.00030 | F=3.4, P=0.017 |

Percent median increase in the first four columns corresponds to the percent difference in each marker between statin users and non-users within each enterotype. P-values in these columns correspond to t-tests comparing covariate adjusted values between statin users and non-users. Values shown are raw p-values, and those that remained significant after correcting for type-1-error (Bonferroni P<0.05) are underlined. The last three columns in the table show the F- and p-values for the statin-by-enterotype interaction term from ANOVA (unadjusted) and ANCOVA (covariate adjusted) models predicting each of the specified markers of glucose homeostasis. Covariate adjusted models were adjusted for microbiome vendor, clinical lab vendor, sex, age, BMI, LDL cholesterol and plasma HMG. ast column corresponds to models adjusted for the same covariates as well as T2D status (yes/no, N=1691, T2D n=64). P-values<0.05 are underlined. Abbreviations: HOMA-IR: Homeostatic Model Assessment for Insulin Resistance; HbA1c: Glycated Hemoglobin Alc.

### Independent Cohort for Evaluating Statin-Microbiome Interactions

To evaluate the robustness of the microbiome associations with markers of statin on-target and adverse effects reported in the cohort, the main results were validated in an independent European cohort of subjects recruited to capture various stages of the cardiometabolic disease spectrum the MetaCardis cohort. Consistent with the original findings, serum HMG was markedly increased in MetaCardis subjects on statins compared to non-statin users, further pointing to its utility as a readily-available biomarker of statin efficacy. Using metagenomics species (MGS) count as a measure of gut α-diversity, a significant MGS count-by-statin interaction effect was observed when predicting serum HMG levels, consistent with the original results (covariate adjusted ANCOVA, P=0.035). Similar to the subjects in the original cohort, MetaCardis subjects with higher gut alpha-diversity demonstrated lower levels of serum HMG compared to subjects with low alpha-diversity, with this relationship being present exclusively in statin users. This interaction was independent of sex, age, BMI, nationality of the participant and microbial load. This sheds some light on potential mechanisms underlying the observed associations, where the primary driver of the observed phenomenon is likely not the difference in the total number of microbes present in the ecosystem, but rather the differences in the taxonomic and functional composition of the gut microbiome.

Given that the MetaCardis study collected stool shotgun metagenomics sequencing data to characterize the gut microbiome, possible functional characteristics of the gut metagenome associated with markers of statin efficacy were explored. To this end, associations between microbiome functions (gut metabolic modules (GMMs) and Kyoto Encyclopedia of Genes and Genomes (KEGG) modules) calculated in the original study, and serum HMG, specifically in statin-users, adjusted for age, sex, BMI, and subject nationality utilizing a beta-binomial regression approach (corncob) were tested. A total of 5 modules remained significantly associated with serum HMG among statin users after multiple-hypothesis correction (Bonferroni P<0.05), including a negative association between HMG and a mucin degradation module.

Statin-dependent associations between gut microbiome enterotypes and measures of statin on-target effects (serum HMG) and adverse effects (Hba1c, the sole marker of glucose homeostasis available in the validation dataset) were evaluated. MetaCardis subjects were separated into four enterotype groups, similar in taxonomic composition to the original cohort, and consistent with previous studies on the same study population. Consistent with previous findings, subjects with ischemic heart disease within the MetaCardis cohort demonstrated a lower likelihood of having a Bac.2 enterotype while on statins (OR(95%CI):0.4 (0.2-0.9),n=303,p=0.022, models adjusted for sex and age). However, non-IHD (i.e., the remainder of the cohort) obese subjects from the MetaCardis cohort demonstrated a trend more consistent with what was observed in the original dataset (e.g., higher odds of Bac.2 enterotype with statin use, adj. OR(95%CI): 1.9(0.8-4.8), P=0.16).

Statin-dependent associations between gut microbiome enterotypes and markers of statin on-target and adverse effects were then validated. There was a significant enterotype-by-statin interaction when modelling serum HMG, independent of age, sex, BMI, nationality, and microbial load, with results strikingly similar to those originally obtained in the original cohort (P=0.035, FIG. 3D, FIG. 4D). Similarly, HbAlc levels were significantly higher in statin users versus non-users across both the Bac.1 and Bac.2 enterotypes, while this increase was absent in the Rum. enterotype. This once again suggests that the risk of metabolic adverse effects may be modulated by a subject’s gut microbiome compositional state. However, the P-value for the interaction term did not reach statistical significance (covariate-adjusted interaction term P=0.195) in the validation cohort, partially due to the smaller sample size compared to the original dataset (Original N=1512, MetaCardis N=688). Because Bac.1 and Bac.2 enterotypes are both enriched for the genus Bacteroides and show similar associations with HbA1c based on statin use, the association between this marker of glycemia and rarefied (e.g., even subsampling of counts without replacement across samples) Bacteroides abundance counts adjusted for total microbial cell count were examined. Consistent with the enterotype analysis, associations between Bacteroides abundance and markers of statin on-target efficacy and metabolic health parameters in statin users were found, which were entirely absent in non-users. Collectively, these results show a high degree of consistency across geographically distinct populations and different gut microbiome sequencing methods (e.g., 16S rRNA amplicon sequencing in the original cohort versus shotgun metagenomic sequencing in the MetaCardis cohort), converging on strong evidence for the potential clinical applicability of the reported findings.

### Discussion

Gut microbiome taxonomic composition can explain interindividual variability in statin responses. There is considerable heterogeneity in response to statin therapy among subjects, both in terms of on-target effects (lowering LDL-cholesterol) and likelihood of experiencing unwanted side-effects. The variation in gut microbiome taxonomic composition can explain interindividual variability in statin responses. The main findings of the analyses are as follows: 1) HMG measured in plasma is a robust marker of both statin use and statin on-target effects, which also reflects known genetic variability in statin responses; 2) Gut alpha-diversity negatively correlates with HMG exclusively in statin users, independent of dose intensity and genetic predisposition, indicating a more diverse microbiome may interfere with statin on-target effects; 3) Enterotype analysis further confirms similar patterns of microbiome modification of statin response, with the Bacteroides dominant, alpha-diversity-depleted Bac.2 enterotype showing the greatest increase in plasma HMG and decrease in LDL-cholesterol levels among statin users; and 4) Of the four enterotypes identified, subjects with the Bac.2 followed by Bac.1 enterotypes experience greatest disruption to glucose control with statin use, while the Firmicutes rich Rum. enterotype appears most protective, indicating variable risk of statin-mediated metabolic side effects based on gut microbiome composition. Collectively, the findings indicate that the gut microbiome influences statin actions. With further refinement, knowledge of these effects may inform statin therapy guidelines and help personalize ASCVD treatment.

The study showed HMG to be a marker of time-invariant monitoring of statin efficacy and off-target effects on metabolic health parameters. The conversion of HMG-CoA to HMG is dependent on the hydrolysis of the thioester bond linking HMG to its Coenzyme-A moiety, which is facilitated by at least one known thioesterase (peroxisomal acyl-CoA thioesterase 2). There are several advantages for including HMG along with LDL-cholesterol measurements when evaluating statin effects. For one, HMG may provide more time-invariant insight into statin efficacy, as opposed to LDL-cholesterol, which requires knowledge of pre-statin cholesterol levels to calculate the percent decrease in LDL over time. This seemed to be the case in the genetics analysis, where cross-sectional measurements of plasma HMG were able to capture genetic variability in statin response while LDL-cholesterol measurements from the same blood draw were less sensitive. In addition, plasma HMG may prove useful when evaluating statin off-target effects on metabolic health parameters, where statistical models can be adjusted for HMG to account for variability in statin on-target effects, as was done in the analysis exploring markers of insulin resistance.

Enterotype is a marker of off-target effects on metabolic health parameters. One finding in the study was an absence of statin-associated metabolic disruption in subjects with a Rum. enterotype (FIG. 5G, FIG. 8). Statin use in this group was still associated with increased plasma HMG and decreased LDL-cholesterol levels (FIG. 5F, FIG. 8), indicating that subjects with this microbiome composition type may benefit from statin therapy without an increased risk of unwanted metabolic complications. There are several possible explanations for this observation. For example, the Rum. enterotype is enriched in the genus Akkermansia, as well as several butyrate-producing taxa, which positively impact host metabolism through multiple mechanisms (Table 4, FIG. 7), potentially serving as a buffer against statin off-target effects on glucose homeostasis. In addition, statin therapies and other prescription medications may be most readily metabolized by species within the Bacteroides genus, of which the Rum. enterotype is most depleted. The lower degree of metabolism by Firmicutes taxa comprising the Rum. enterotype may therefore be potentially protective from statin off-target effects. Consistently, both Bacteroides rich Bac.1 and Bac.2 enterotypes showed greatest increases in markers of insulin resistance with statin use.

Statin use in subjects with the Bac.2 enterotype was associated with the strongest on-target effects (e.g., increase in plasma HMG and decrease in LDL-cholesterol) but also greatest metabolic disruption among all four enterotypes (FIGS. 5F-G, FIG. 8). This is consistent with the identified association between the magnitude of decrease in LDL-cholesterol with statin use and risk of developing T2D (e.g., the greater the percent decrease in LDL-cholesterol with statin therapy, the higher the risk of new onset T2D). One possible mechanism behind the reported association is the previously mentioned ability of Bacteroides species to metabolize prescription medications, including statin therapies. Bacteroides dominance within both the Bac.1 and Bac.2 enterotypes may modify drug activity, impacting both potency and potential side effects. Paired with depletion of several major butyrate-producing taxa within the Bac.2 enterotype (FIG. 5D, FIG. 5, Table 4), this bacterial composition may put subjects at particularly high risk of metabolic complications. If this were indeed the case, subjects with a Bac.2 enterotype could benefit most from lower intensity therapy, which may achieve the desired percent decrease in LDL-cholesterol while mitigating potential metabolic disruptions. Complementary probiotic and prebiotic interventions could also be potentially pursued in these subjects.

Referring to FIG. 8, microbiome enterotypes are shown to modify markers of statin on-and off-target effects. Graph 800A depicts blood LDL-cholesterol levels among statin users and non-users stratified by enterotype. Interaction P corresponds to the statin-by-enterotype interaction term P-value from unadjusted ANOVA models, while the cov. Adj. interaction P corresponds to the statin-by-enterotype interaction term P-value from ANCOVA models adjusted for clinical lab vendor, microbiome vendor, sex, age, BMI and LDL cholesterol. Values shown on the y-axis are values adjusted for the same covariates (residuals). Graph 800B shows HbA1c measures among statin users and non-users stratified by enterotype. Interaction P corresponds to an unadjusted interaction term P-value as in graph 800A, while the cov. Adj. interaction P corresponds to the statin-by-enterotype interaction term P-value from ANCOVA models adjusted for clinical lab vendor, microbiome vendor, sex, age, BMI, HMG and LDL cholesterol. Values shown on the y-axis are values adjusted for the same covariates (residuals). P-values above the box plots across graphs 800A-B correspond to tests of significance between statin non-users and statin users within each enterotype using two-samples t-test on covariate adjusted values (residuals). Differences with Bonferroni corrected P<0.05 were considered statistically significant. Box plots represent the interquartile range (25th to 75th percentile, IQR), with the middle line denoting the median; whiskers span 1.5 × IQR, points beyond this range are shown individually.

The analyses indicate that statins have a detectable, but weak effect on the composition of the gut microbiome, while the gut microbiome appears to have a more sizable impact on host responses to statin therapy.

### Prediction of Gut Microbiome Composition From Blood Metabolomics Data

Having demonstrated that statin therapy intensity with reduced risks of side effects can be predicted directly from gut microbiome diversity and abundance data, the study set out to examine whether blood metabolite data could be used for this purpose. The objective was to test the ability of blood markers to indirectly predict gut microbiome composition, and then use that output to predict statin therapy intensity having the gut microbiome influence built into the result.

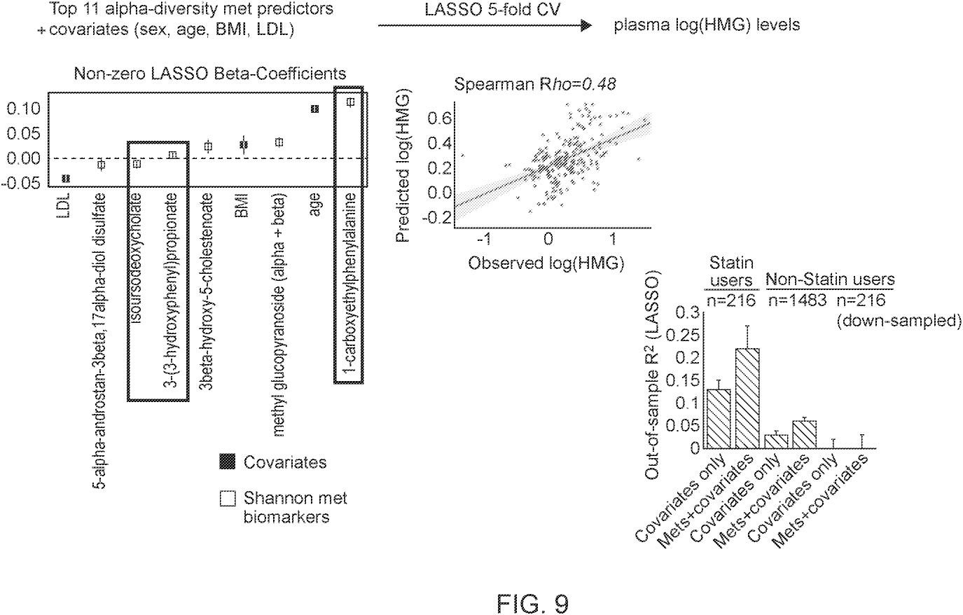

Initially, a Least Absolute Shrinkage and Selection Operator (“LASSO”) was applied to 11 metabolites shown to be predictive of gut alpha-diversity. The study examined HMG as a surrogate output for this purpose since gut alpha-diversity was found to negatively correlate with HMG exclusively in statin users. The results are shown in FIG. 9. As can be seen in FIG. 9, up to about 22-25% of the variance can be explained using a conservative 5-fold cross-validation scheme, with most of the alpha-diversity signal accounted for by Bac.2 subjects. While this can be improved by controlling for Bac.2, the study endeavored to predict Bac.2 enterotype from blood metabolite data.

Referring to FIG. 9, Shannon diversity biomarkers are shown to predict HMG levels exclusively in statin users. A total of 11 plasma metabolites identified as strong predictors of gut microbiome Shannon diversity were used, as well as LDL-cholesterol, BMI, and age, to predict plasma HMG levels using a penalized regression machine learning algorithm (LASSO). The beta-coefficients from the model are shown, with metabolites denoted by the white boxes. The boxes highlight metabolites that are strictly microbial (not produced by the host, but rather a result of microbial metabolism). The scatterplot shows the relationship between out-of-sample (test set) predicted HMG levels versus observed (actual) HMG values for statin users. The bar plots to the right show the model performance in predicting HMG levels in statin users and non-users. The metabolite models predicting HMG work only in statin users.

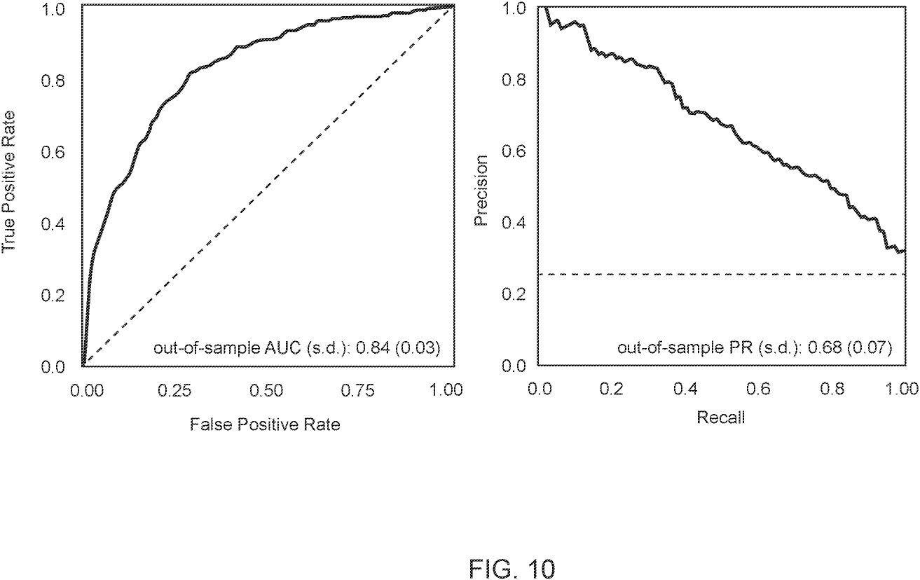

The Bac.2 enterotype encompasses about 25% of the subjects examined. To account for the lower number of cases than controls, a 10-fold cross validation (“CV”) implementation of Random Forests with a weight parameter was applied to the data. Performance was then evaluated across the 10-fold CV using each fold as a test-set. The results are shown in Table 5 and in FIG. 10, which depicts the associated Precision-Recall and ROC curves. As can be seen, a decent signal (AUC =~0.84) is observed. Additional blood metabolite panels and artificial-intelligence algorithm selection may improve the signal since the Metabolon panel applied in this study represents a subset of plasma metabolites. These results demonstrate that machine learning classifiers can be constructed to predict gut microbiome-dependent statin therapy intensity from blood metabolite data.

FIG. 10 shows blood metabolomics data predict Bacteroides 2 enterotype. Receiver operator characteristic (ROC) and precision-recall (PR) curves for test-set predictions of whether a subject has the Bac.2 enterotype or any of the other three enterotypes are shown. A random-forest machine learning classifier was trained on plasma metabolomics data and evaluated using a 10-fold cross-validation scheme. The dashed line shows the performance of a completely random prediction.

TABLE 5

Results from 10-fold CV out-of-sample performance

| | |
|---|---|
| mean sensitivity | 0.5278002699055331 |
| mean specificity | 0.8998680329141437 |
| mean precision | 0.6493485686997708 |
| mean PR AUC | 0.6737018564248644 |
| std dev. | 0.06753517273706344 |
| mean ROC AUC | 0.8357641750166204 |
| std dev ROC AUC | 0.028567451303754005 |

### Statin Therapy Intensity Scoring

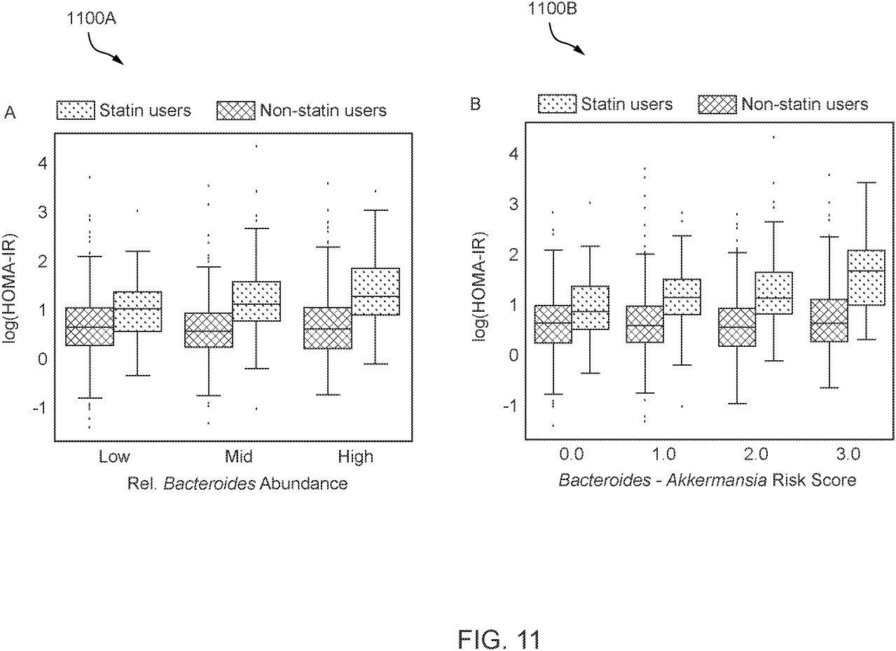

The study next set out to apply the findings in a statin therapy intensity scoring application. Initial models utilized interpretable rule-based classification with an adjustable quantile scoring strategy that considered both statin efficacy and risk of insulin resistance side-effects in the output. The models also included adjustments to account for other attributes, such as genetic markers associated with insulin resistance, cardio-metabolic gut commensals, and the like. The presence or absence of the cardio-metabolic gut commensal Akkermansia in the Bacteroides abundance analysis was chosen as a test case. The results are shown in FIG. 11 and Tables 6 and 7.

FIG. 11 shows Bacteroides abundance predicts insulin resistance features levels exclusively in statin users, and that the presence or absence of Akkermansia can be a part of an insulin resistance risk score. Graph 1100A depicts log transformed HOMA-IR levels in statin users and non-users across low (<11.5%), mid (11-5%-21%) and high (<21%) levels of Bacteroides. Relative Bacteroides abundance was measured via a stool sample and 16SrRNA amplicon gene sequencing. Taxonomy assignment of ASVs was performed using the RDP classifier with the SILVA database. The count matrix was further rarefied to an even sampling depth of 22500 reads. HOMA-IR levels were calculated using blood insulin and glucose levels. Graph 100B shows log transformed HOMA-IR levels in statin users and non-users across a combined Bacteroides - Akkermansia risk score (e.g.., Bacteroides abundance without the presence of Akkermansia). Relative Bacteroides and Akkermansia abundance was obtained using the same methodology as in graph 1100A.

Graph 1100A shows the risk of insulin resistance as measured by HOMA-IR increases with Bacteroides abundance and occurs exclusively in statin users. Graph 1100B that adjusting for Akkermansia by computationally simulating its absence from the dataset impacts the insulin risk score, in this example, by one unit. The impact was incorporated as an adjustment to the statin therapy intensity score, for example, as illustrated in the scoring models graphically depicted in Tables 6 and 7. As can be seen in Tables 6 and 7, the Akkermansia adjustment can be combined with additional adjustment features to account for high intensity statin therapy in combination with monitoring and/or treating for insulin resistance when cardiovascular treatment at higher statin level outweighs the risk of side-effects. A more conservative model is depicted in Table 7, which attributes a smaller impact on baseline statin therapy intensity prediction from adjustment features such as Akkermansia, monitoring and/or treating for insulin resistance. Fractional scores from the more conservative can be rounded up to approximate the less conservative model.

TABLE 6

Statin Therapy Intensity Model 1

| Rx intensity (score) | High (0-1) | Moderate (1-2) | | Low (2-3) | |
|---|---|---|---|---|---|
| Bacteroides abundance | 0% | 11.5% | 21.0% | | |
| Rx intensity score IR Rx-, Akk- | 0.0 | 1.0 | 2.0 | | 3.0 |
| IR Rx-, Akk+ | 0.0 | 0.0 | 1.0 | | 2.0 |
| IR Rx+, Akk- | 0.0 | 0.0 | 1.0 | | 2.0 |
| IR Rx+, Akk+ | 0.0 | 0.0 | 0.0 | | 1.0 |
| Alpha-diversity (SI) | ← | 4.47 | 4.14 | | 0.00 |
| Rx intensity score IR Rx- | 0.0 | 1.0 | 2.0 | | 3.0 |
| IR Rx+ | 0.0 | 0.0 | 1.0 | | 2.0 |
| Enterotype assignments | Rum. or Prev. | | Bac.1 | | Bac.2 |
| Rx intensify score IR Rx- | 0.0 | 1.0 | 2.0 | | 3.0 |
| IR Rx+ | 0.0 | 0.0 | 1.0 | | 2.0 |

TABLE 7

Statin Therapy Intensity Model 2

| Rx intensity (score) | | High (0-1) | | Moderate (1-2) | | | Low (2-3) | |
|---|---|---|---|---|---|---|---|---|
| Batcteroides abundance | | 0% | | 11.5% | | 21.0% | → | |
| Rx intensity score | IR Rx-, Akk- | 0.0 | 0.5 | 1.0 | 1.5 | 2.0 | 2.5 | 3.0 |
| | IR Rx-, Akk+ | 0.0 | 0.0 | 0.5 | 1.0 | 1.5 | 2.0 | 2.5 |
| | IR Rx+, Akk- | 0.0 | 0.0 | 0.5 | 1.0 | 1.5 | 2.0 | 2.5 |
| | IR Rx+, Akk+ | 0.0 | 0.0 | 0.0 | 0.5 | 1.0 | 1.5 | 2.0 |

| | | | | | | | | |
|---|---|---|---|---|---|---|---|---|
| Alpha-diversity (SI) | | ← | | 4.47 | | 4.14 | | 0.00 |
| Rx intensity score | IR Rx- | 0.0 | 0.5 | 1.0 | 1.5 | 2.0 | 2.5 | 3.0 |
| | IR Rx+ | 0.0 | 0.0 | 0.5 | 1.0 | 1.5 | 2.0 | 2.5 |

| | | | | | | | | |
|---|---|---|---|---|---|---|---|---|
| Enterotype assignments | Rum or Prev | | | | Bac1 | | Bac2 | |
| Rx intensity score | IR Rx- | 0.0 | 0.5 | 1.0 | 1.5 | 2.0 | 2.5 | 3.0 |
| | IR Rx+ | 0.0 | 0.0 | 0.5 | 1.0 | 1.5 | 2.0 | 2.5 |

Rx intensity scores: 0 = high intensity statin therapy; 1 = moderate or high intensity statin therapy; 2 = moderate or low intensity statin therapy; 3 = low intensity statin therapy.
Abbreviations: Rx intensity = statin therapy intensity; IR Rx = insulin resistance monitoring and/or treatment; Bac = Bacteroides; Rum = Ruminococacceae; Prev = Prevotella; Akk = Akkermansia; SI = Shannon Index.

### Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed:

1. A computer-implemented method comprising:
(a) accessing gut compositional data including a taxonomic abundance, a taxonomic diversity, and/or an enterotype for a subject;
(b) generating a gut microbiome signature for a safety of a statin therapy for the subject and an efficacy of the statin therapy for the subject by applying a classifier to the gut compositional data, the safety of the statin therapy characterized by an insulin resistance of the subject, and the efficacy of the statin therapy characterized by a blood hydroxymethylglutarate (HMG) level of the subject;
(c) determining a recommended therapy for the subject based on the gut microbiome signature and one or more taxa of the gut compositional data of the subject, the recommended therapy selected from a statin therapy intensity, a probiotic therapy, a prebiotic therapy, or a combination thereof; and
(d) outputting the recommended therapy.

2. The computer-implemented method of claim 1, wherein determining the recommended therapy comprises:
comparing the gut microbiome signature and the gut compositional data of the subject to a reference dataset, the reference dataset comprising a plurality of gut microbiome data and blood metabolite data of a reference population exhibiting variable insulin resistance and blood HMG level responses to a given statin therapy intensity.

3. The computer-implemented method of claim 1, further comprising:
determining a presence of Akkermansia for the subject is below a first threshold based on the gut compositional data; and
facilitating the probiotic therapy and/or the prebiotic therapy for the subject based on the presence of Akkermansia being below the first threshold.

4. The computer-implemented method of claim 1, further comprising:
determining the blood HMG level for the subject; and
generating the gut microbiome signature for the subject by applying the classifier to the gut compositional data and the blood HMG level.

5. The computer-implemented method of claim 1, further comprising:
accessing fecal nucleic acid sequence data and/or blood metabolite data for the subject; and
generating the gut compositional data for the subject based on the fecal nucleic acid sequence data and/or the blood metabolite data.

6. The computer-implemented method of claim 1, wherein determining the recommended therapy comprises one or more steps selected from:
determining the gut compositional data includes a relative abundance of Bacteroides ssp. above a first threshold for the subject;
determining that the enterotype included in the gut compositional data is a Bacteroides 1 enterotype or a Bacteroides 2 enterotype;
determining the gut compositional data includes an alpha-diversity below a second threshold for the subject; and
determining the statin therapy intensity is below a threshold intensity.

7. The computer-implemented method of claim 1, wherein determining the recommended therapy comprises one or more steps selected from:
determining the gut compositional data includes a relative abundance of Bacteroides ssp. above a first threshold for the subject;
determining that the enterotype included in the gut compositional data is a Bacteroides 1 enterotype or a Bacteroides 2 enterotype;
determining the gut compositional data includes an alpha-diversity below a second threshold for the subject;
determining at least one of: (i) a presence of Akkermansia for the subject, (ii) an insulin resistance characterization for the subject, or (iii) a treatment for insulin resistance for the subject; and
determining the statin therapy intensity is above a threshold intensity.

8. The computer-implemented method of claim 1, wherein determining the recommended therapy comprises one or more steps selected from:
determining the gut compositional data includes a relative abundance of Bacteroides ssp. below a first threshold for the subject;
determining that the enterotype indicated by the gut compositional data excludes a Bacteroides enterotype;
determining the gut compositional data includes an alpha-diversity greater than a second threshold for the subject; and
determining the statin therapy intensity is greater than a threshold intensity.

9. The computer-implemented method of claim 1, further comprising:
determining a genetic risk score associated with the subject having one or more alleles associated with the efficacy of the statin therapy for the subject or the safety of the statin therapy for the subject; and
generating the gut microbiome signature for the subject by applying the classifier to the gut compositional data and the genetic risk score.

10. A system comprising:
one or more data processors; and
a non-transitory computer readable storage medium containing instructions which, when executed on the one or more data processors, cause the one or more data processors to perform a set of actions including:
(a) accessing gut compositional data including a taxonomic abundance, a taxonomic diversity, and/or an enterotype for a subject;
(b) generating a gut microbiome signature for a safety of a statin therapy for the subject and an efficacy of the statin therapy for the subject by applying a classifier to the gut compositional data, the safety of the statin therapy characterized by an insulin resistance of the subject, and the efficacy of the statin therapy characterized by a blood hydroxymethylglutarate (HMG) level of the subject;
(c) determining a recommended therapy for the subject based on the gut microbiome signature and one or more taxa of the gut compositional data of the subject, the recommended therapy selected from a statin therapy intensity, a probiotic therapy, a prebiotic therapy, or a combination thereof; and
(d) outputting the recommended therapy.

11. The system of claim 10, wherein the set of actions further include determining the recommended therapy by:
comparing the gut microbiome signature and the gut compositional data of the subject to a reference dataset, the reference dataset comprising a plurality of gut microbiome data and blood metabolite data of a reference population exhibiting variable insulin resistance and blood HMG level responses to a given statin therapy intensity.

12. The system of claim 10, wherein the set of actions further includes:
determining a presence of Akkermansia for the subject is below a first threshold based on the gut compositional data; and
facilitating the probiotic therapy and/or the prebiotic therapy for the subject based on the presence of Akkermansia being below the first threshold.

13. The system of claim 10, wherein the set of actions further includes:
determining the blood HMG level for the subject; and
generating the gut microbiome signature for the subject by applying the classifier to the gut compositional data and the blood HMG level.

14. The system of claim 10, wherein the set of actions further includes:
accessing fecal nucleic acid sequence data and/or blood metabolite data for the subject; and
generating the gut compositional data for the subject based on the fecal nucleic acid sequence data and/or the blood metabolite data.

15. The system of claim 10, wherein the set of actions further includes determining the recommended therapy by performing one or more steps selected from:
determining the gut compositional data includes a relative abundance of Bacteroides ssp. above a first threshold for the subject;
determining that the enterotype included in the gut compositional data is a Bacteroides 1 enterotype or a Bacteroides 2 enterotype;
determining the gut compositional data includes an alpha-diversity below a second threshold for the subject; and
determining the statin therapy intensity is below a threshold intensity.

16. The system of claim 10, wherein the set of actions further includes determining the recommended therapy by performing one or more steps selected from:
determining the gut compositional data includes a relative abundance of Bacteroides ssp. above a first threshold for the subject;
determining that the enterotype included in the gut compositional data is a Bacteroides 1 enterotype or a Bacteroides 2 enterotype;
determining the gut compositional data includes an alpha-diversity below a second threshold for the subject;
determining at least one of: (i) a presence of Akkermansia for the subject, (ii) an insulin resistance characterization for the subject, or (iii) a treatment for insulin resistance for the subject; and
determining the statin therapy intensity is above a threshold intensity.

17. The system of claim 10, wherein the set of actions further includes determining the recommended therapy by performing one or more steps selected from:
determining the gut compositional data includes a relative abundance of Bacteroides ssp. below a first threshold for the subject;
determining that the enterotype indicated by the gut compositional data excludes a Bacteroides enterotype;
determining the gut compositional data includes an alpha-diversity greater than a second threshold for the subject; and
determining the statin therapy intensity is greater than a threshold intensity.

18. The system of claim 10, wherein the set of actions further include:
determining a genetic risk score associated with the subject having one or more alleles associated with the efficacy of the statin therapy for the subject or the safety of the statin therapy for the subject; and
generating the gut microbiome signature for the subject by applying the classifier to the gut compositional data and the genetic risk score.

19. A computer-program product tangibly embodied in a non-transitory machine-readable storage medium, including instructions configured to cause one or more data processors to perform a set of actions including:
(a) accessing gut compositional data including a taxonomic abundance, a taxonomic diversity, and/or an enterotype for a subject;
(b) generating a gut microbiome signature for a safety of a statin therapy for the subject and an efficacy of the statin therapy for the subject by applying a classifier to the gut compositional data, the safety of the statin therapy characterized by an insulin resistance of the subject, and the efficacy of the statin therapy characterized by a blood hydroxymethylglutarate (HMG) level of the subject;
(c) determining a recommended therapy for the subject based on the gut microbiome signature and one or more taxa of the gut compositional data of the subject, the recommended therapy selected from a statin therapy intensity, a probiotic therapy, a prebiotic therapy, or a combination thereof; and
(d) outputting the recommended therapy.

20. The computer-program product of claim 19, wherein the set of actions further include determining the recommended therapy by:
comparing the gut microbiome signature and the gut compositional data of the subject to a reference dataset, the reference dataset comprising a plurality of gut microbiome data and blood metabolite data of a reference population exhibiting variable insulin resistance and blood HMG level responses to a given statin therapy intensity.

21. The computer-program product of claim 19, wherein the set of actions further includes:
determining a presence of Akkermansia for the subject is below a first threshold based on the gut compositional data; and
facilitating the probiotic therapy and/or the prebiotic therapy for the subject based on the presence of Akkermansia being below the first threshold.

22. The computer-program product of claim 19, wherein the set of actions further includes:
determining the blood HMG level for the subject; and
generating the gut microbiome signature for the subject by applying the classifier to the gut compositional data and the blood HMG level.

23. The computer-program product of claim 19, wherein the set of actions further includes:
accessing fecal nucleic acid sequence data and/or blood metabolite data for the subject; and
generating the gut compositional data for the subject based on the fecal nucleic acid sequence data and/or the blood metabolite data.

24. The computer-program product of claim 19, wherein the set of actions further includes determining the recommended therapy by performing one or more steps selected from:
determining the gut compositional data includes a relative abundance of Bacteroides ssp. above a first threshold for the subject;
determining that the enterotype included in the gut compositional data is a Bacteroides 1 enterotype or a Bacteroides 2 enterotype;
determining the gut compositional data includes an alpha-diversity below a second threshold for the subject; and
determining the statin therapy intensity is below a threshold intensity.

25. The computer-program product of claim 19, wherein the set of actions further includes determining the recommended therapy by performing one or more steps selected from:
determining the gut compositional data includes a relative abundance of Bacteroides ssp. above a first threshold for the subject;
determining that the enterotype included in the gut compositional data is a Bacteroides 1 enterotype or a Bacteroides 2 enterotype;
determining the gut compositional data includes an alpha-diversity below a second threshold for the subject;
determining at least one of: (i) a presence of Akkermansia for the subject, (ii) an insulin resistance characterization for the subject, or (iii) a treatment for insulin resistance for the subject; and
determining the statin therapy intensity is above a threshold intensity.

26. The computer-program product of claim 19, wherein the set of actions further includes determining the recommended therapy by performing one or more steps selected from:
determining the gut compositional data includes a relative abundance of Bacteroides ssp. below a first threshold for the subject;
determining that the enterotype indicated by the gut compositional data excludes a Bacteroides enterotype;
determining the gut compositional data includes an alpha-diversity greater than a second threshold for the subject; and
determining the statin therapy intensity is greater than a threshold intensity.

27. The computer-program product of claim 19, wherein the set of actions further include:
determining a genetic risk score associated with the subject having one or more alleles associated with the efficacy of the statin therapy for the subject or the safety of the statin therapy for the subject; and
generating the gut microbiome signature for the subject by applying the classifier to the gut compositional data and the genetic risk score.
